(19) United States
(12) Patent Application Publication
(10) Pub. No.: US 2024/0065617 A1
(43) Pub. Date: Feb. 29, 2024

(54) Digital Apparatus and Application for Arthritis Therapy and Methods of Use Thereof

(71) Applicant: S-ALPHA THERAPEUTICS, INC., Seoul (KR)

(72) Inventor: Seung Eun Choi, Seoul (KR)

(73) Assignee: S-ALPHA THERAPEUTICS, INC., Seoul (KR)

(21) Appl. No.: 18/272,036

(22) PCT Filed: Jan. 11, 2022
(86) PCT No.: PCT/KR2022/000481

§ 371 Date: Jul. 12, 2023

Related U.S. Application Data
(60) Provisional application No. 63/136,320, filed on Jan. 12, 2021.

(51) Int. Cl. A61B 5/00; G16H 20/30

(52) U.S. Cl. CPC A61B 5/4528; G16H 20/30; A61M 2021/0016

(57) ABSTRACT

Systems and methods for arthritis therapy are provided. A system may include a digital apparatus, which may include a digital instruction generation unit configured to generate digital therapeutic modules for preventing or ameliorating arthritis, generate specified digital instructions based on the digital therapeutic modules and provide the digital instructions to a first user, and an outcome collection unit configured to collect the first user's execution outcomes of the digital instructions.

### TECHNICAL FIELD

The present disclosure relates to digital therapeutics (hereinafter referred to as DTx) intended for arthritis therapy. The present disclosure also relates to systems that integrate digital therapeutics with one or both of a healthcare provider portal and an administrative portal to prevent or ameliorate arthritis in a patient. Some embodiments of the present disclosure may comprise deducing a mechanism of action (hereinafter referred to as MOA) in arthritis, and establishing a therapeutic hypothesis and a digital therapeutic hypothesis for preventing and/or ameliorating arthritis in a patient based on these findings.

### BACKGROUND ART

Arthritis is characterized by joint inflammation, acute or chronic pain, and in some cases irreversible damage of tendons, ligaments, joints, and bones throughout the body. Current therapies for arthritis include medication aimed at reducing pain or inflammation of the joint. However, such treatment (e.g., using small molecules, biologics, etc.) is often focused on pain management, and not treating the underlying condition or prophylaxis. In particular, existing treatments fail to prevent or ameliorate joint inflammation before chronic inflammation causes irreversible damage. A common course of treatment for reducing inflammation includes the prescription of nonsteroidal anti-inflammatories (NSAIDs) or corticosteroids. However, long term use of high-doses of NSAIDs and corticosteroids can have profound side effects. Thus, there is a need in the art for alternative therapies with better risk-benefit profiles (e.g., digital therapeutics) for preventing and/or ameliorating arthritis.

### DISCLOSURE OF INVENTION

### Brief Description of Drawings

The above and other objects, features and advantages of the present disclosure will become more apparent to those of ordinary skill in the art by describing in detail exemplary embodiments thereof with reference to the attached drawings, in which:

FIG. 1 illustrates various biochemical pathways and physiological symptoms associated with arthritis, as well as applications of certain embodiments of the present disclosure and associated effects;

FIG. 2 illustrates the mechanism of action, including various biochemical factors, associated with the post-operative care of the knee;

FIG. 3 is a table showing an exemplary strategy for developing embodiments of the present disclosure, including a review of the state of the art, regulatory challenges, data gathering with clinical trials for showing efficacy of treatment and safety of the medical device, and identifying the target population.

FIG. 4 is a block diagram showing an exemplary configuration of a digital apparatus for preventing and/or ameliorating arthritis according to one embodiment of the present disclosure;

FIG. 5 is a diagram showing exemplary input and output loops of a digital application for preventing and/or ameliorating arthritis according to one embodiment of the present disclosure;

FIG. 6 is a diagram showing an exemplary feedback loop for a digital apparatus and an application for preventing and/or ameliorating arthritis according to one embodiment of the present disclosure;

FIG. 7 is a diagram showing an exemplary background factors supporting the digital apparatus and the application for preventing and/or ameliorating arthritis according to one embodiment of the present disclosure;

FIG. 8 is a diagram showing an exemplary method of assigning a patient-customized digital prescription using the digital apparatus and the application for preventing and/or ameliorating arthritis according to one embodiment of the present disclosure;

FIG. 9 is a flowchart illustrating exemplary operations in a digital application for preventing and/or ameliorating arthritis according to one embodiment of the present disclosure;

FIG. 10 is a diagram showing an exemplary hardware configuration of the digital apparatus for preventing and/or ameliorating arthritis according to one embodiment of the present disclosure;

FIG. 11 is a flow chart illustrating an exemplary system for preventing and/or ameliorating arthritis, the system comprising an administrative portal (e.g., Administrator's web), a healthcare provider portal (e.g., Doctor's web) and a digital apparatus configured to execute a digital application (e.g., an application or ‘app’) for preventing and/or ameliorating arthritis in a subject;

FIG. 12A-G show (A) a flow chart of the login procedure and application usage for a subject, (B) a flow chart illustrating an exemplary execution flow for a login verification during a splash process at the starting of a digital application of the present disclosure, (C) a flow chart illustrating an exemplary execution flow for a prescription verification during a splash process at the starting of a digital application of the present disclosure, (D) a flow chart illustrating exemplary execution flow of a schedule of modules to be performed, and performance of the scheduled module, (E) a flow chart of the application execution flow from starting the application through entry to the home screen, (F) a flow chart of the application execution flow from the home screen through the start of providing one or more modules to the subject, and (G) a flow chart of the application execution flow from the start of providing one or more modules to the subject through completion of the scheduled modules;

FIG. 13A-B depict (A) a calibration screen of a calibration module of a digital application of the present disclosure, wherein the calibration screen indicates a position that the subject's joint (e.g., a knee) should be positioned within a camera's field of view, and (B) a flow chart of the application execution flow for a calibration module of the present disclosure;

FIG. 14A-E depict an execution flow for an joint exercise module of the present disclosure;

FIG. 15A-E depict an execution flow for a benchmark exercise module of the present disclosure;

FIG. 16A-B depict a schedule screen of a digital application of the present disclosure, wherein the schedule screen indicates a schedule of modules available for the subject to perform over a predetermined period of time;

FIG. 17A-L depict (A) a flow chart of the application execution flow for a vagal nerve stimulation module of the present disclosure, and (B-L) various screens for selecting and performing exercises for inducing vagal nerve stimulation;

FIG. 18 depicts a screenshot of a Parent Section in an exemplary digital application of the present disclosure;

FIG. 19 is a flow chart illustrating an exemplary execution flow for a healthcare provider portal in a system of the present disclosure;

FIGS. 20A-E depict (A) an exemplary patient tab in a healthcare provider portal, the patient tab displaying detailed information on a given patient, (B) an exemplary patient tab in a healthcare provider portal that displays detailed prescription information for a given patient, (C-D) an exemplary patient tab in a healthcare provider portal for editing prescription information for a given patient, and (E) a patient tab in a healthcare provider portal for viewing details (e.g., date, status, duration, results) of a given session for a given patient;

FIG. 21 is a flow chart illustrating an exemplary execution flow for an administrative portal in a system of the present disclosure;

FIGS. 22A-E depict (A) an exemplary doctor tab in an administrative portal, the doctor tab displaying a list of doctors, (B) an exemplary doctor tab in an administrative portal, the doctor tab displaying a list of patients being cared for by a given doctor, with patient-identifying information redacted (*), (C) an exemplary patient tab in an administrative portal that displays information for one or more patients, wherein sensitive information is redacted, (D) an exemplary patient tab in an administrative portal that displays detailed patient or prescription information for a given patient, (E) an exemplary patient tab in an administrative portal that displays detailed prescription information for a given patient, and (F) a patient tab in an administrative portal for viewing details (e.g., date, status, duration, results) of a given session for a given patient;

FIGS. 23A-G illustrates various application functions, including modules that may be performed by the subject, for example, to relieve pain symptoms, prevent recurrence of pain/disease, strengthen a joint, and/or induce relaxation; and

FIG. 24 illustrates an exemplary deep breathing exercise of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### MODE FOR THE INVENTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may mean a range of ±20%, ±10%, or ±5% of a given numeric value.

As used herein, “arthritis” can refer to any joint disorder or condition that involves inflammation of one or more joints. The term “arthritis,” as used herein, encompasses a variety of types and subtypes of arthritis of various etiologies and causes, either known or unknown, including, but not limited to, rheumatoid arthritis, osteoarthritis, infectious arthritis, psoriatic arthritis, gouty arthritis, and lupus-related arthritis.

Overview

With reference to the appended drawings, exemplary embodiments of the present disclosure will be described in detail below. To aid in understanding the present disclosure, like numbers refer to like elements throughout the description of the figures, and the description of the same elements will be not reiterated.

Digital therapeutics are realized using software. That is, the core of the digital therapeutics as in the present disclosure depends on the rational design of digital therapeutics suitable for treatment of a corresponding disease, and the development of specific software capable of clinically verifying the digital therapeutics based on the rational design. Hereinafter, a digital apparatus and an application for preventing and/or ameliorating arthritis according to the present disclosure realized in this aspect will be described in detail.

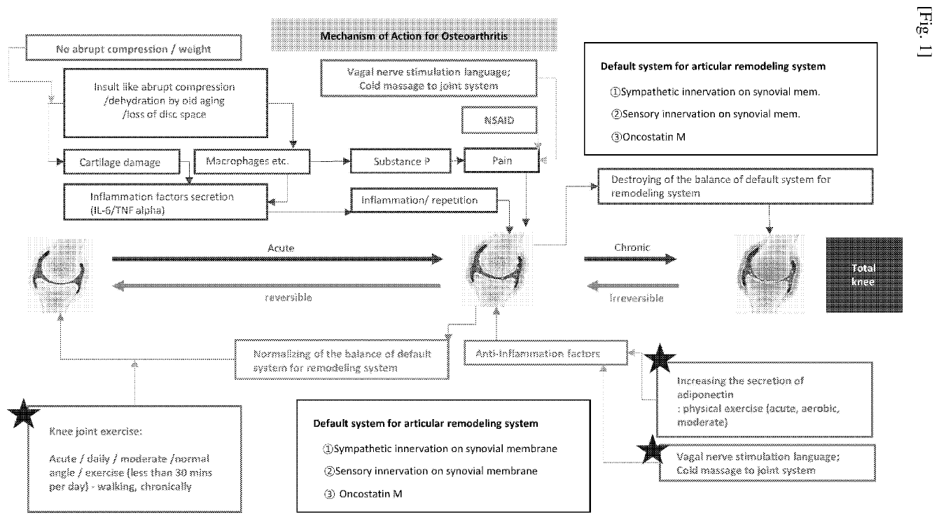

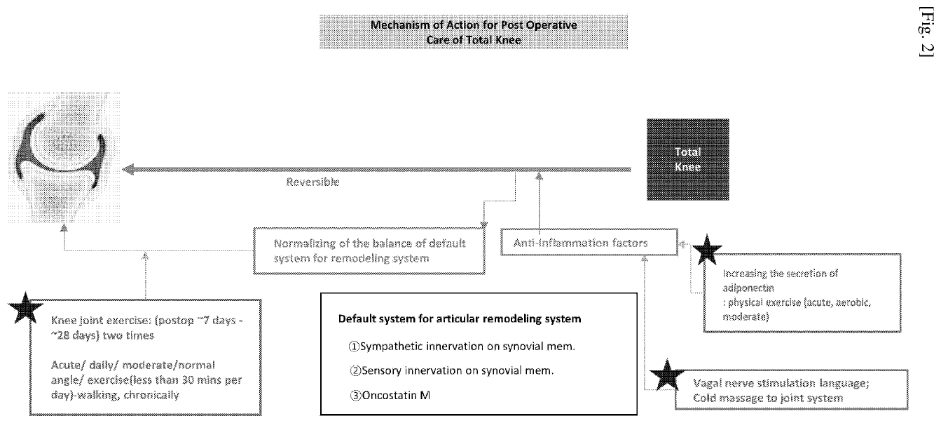

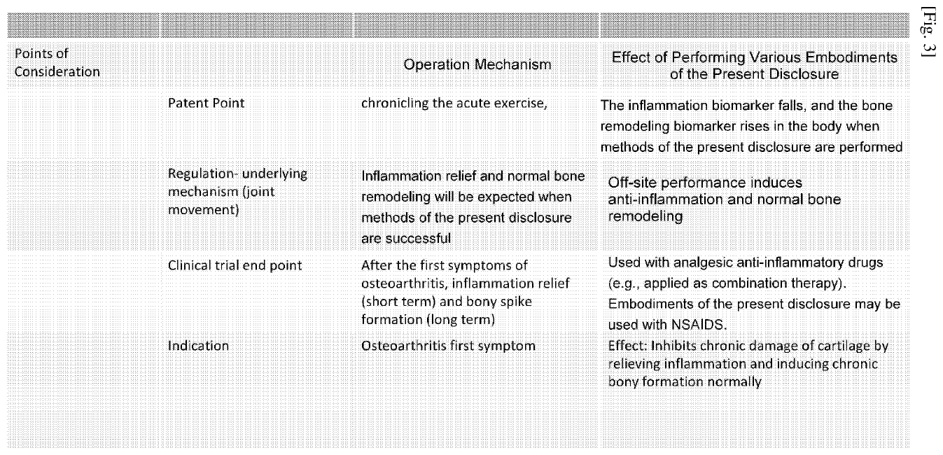

FIG. 1 illustrates various exemplary biochemical pathways and physiological symptoms associated with joint inflammation and/or arthritis, as well as exemplary applications of certain embodiments of the present disclosure, including the use of digital therapeutics to provide a subject with digital therapeutic modules comprising instructions, and associated effects.

As shown in FIG. 1, development and progression of osteoarthritis may occur when a joint experiences insults such as an abrupt force or compression, dehydration due to aging, or loss of disc space. These insults can lead to cartilage damage, the secretion of inflammation factors, such as IL-6 and/or TNF-α, which can then cause acute inflammation of the joint accompanied by pain. When the joint is repeatedly inflamed, the balance of the default system (e.g., the balance between sympathetic innervation on the synovial membrane, sensory innervation on the synovial membrane, and the level of Oncostatin M) for joint remodeling can be disrupted, thereby causing irreversible damage to the joint and chronic inflammation and pain. Vagal nerve stimulation and/or the prescription of NSAIDs can be used to mitigate pain.

FIG. 2 illustrates the mechanism of action, including various biochemical factors, associated with the post-operative care of the knee, and how the digital therapeutic modules of the present disclosure may be used to reverse joint inflammation and damage. In particular, vagal nerve stimulation and adiponectin secretion modules can be used to increase the amount of anti-inflammation factors in the joint. Additionally, a joint exercise module can be used to normalize the balance of the default system for joint remodeling.

As mentioned above, one aim of the methods and systems of the present disclosure is to at least partially reduce joint inflammation and prevent inflammation from causing chronic or irreversible damage to the joint. In one aspect, methods and systems of the present disclosure inhibit activation of the NF-kB pathway by promoting adiponectin secretion, for example, through meditation and/or aerobic exercise. In another aspect, methods and systems of the present disclosure inhibit activation of the NF-kB pathway by inhibiting inflammatory cytokines TNF-α/IL-1 through vagal nerve stimulation.

FIG. 3 provides is a table showing an exemplary strategy for developing embodiments of the present disclosure, including a review of the state of the art, regulatory challenges, data gathering with clinical trials for showing efficacy of treatment and safety of the medical device, and identifying the target population.

A digital apparatus and an application for preventing and/or ameliorating arthritis according to the present disclosure will be described below.

Generally speaking, disease therapy is carried out by analyzing a certain disease in terms of pathophysiological functions and dispositions in order to determine a start point, a progression point, and an end point for the disease. Also, an indication of the disease is defined by characterization of the corresponding disease and statistical analysis of the disease. A patient's physiological factors, especially biochemical factors, which correspond to the verified indications, are analyzed, and the patient's biochemical factors are analyzed to disease to deduce a mechanism of action.

Next, a therapeutic hypothesis, in which the corresponding disease is treated by controlling actions and environments directly associated with regulation of the corresponding biochemical factors associated with the disease, is deduced. To realize this therapeutic hypothesis into digital therapeutics, a digital therapeutic hypothesis for achieving a therapeutic effect through repeated digital instruction and execution, which are associated with the control of patient's action/environment→regulation of biochemical factors, is proposed. digital therapeutic hypothesis of the present disclosure is realized as a digital apparatus and an application is realized as a digital apparatus and an application configured to present changes in patient's actions, and patient's participation in the form of specific instructions and collect and analyze execution of the specific instructions.

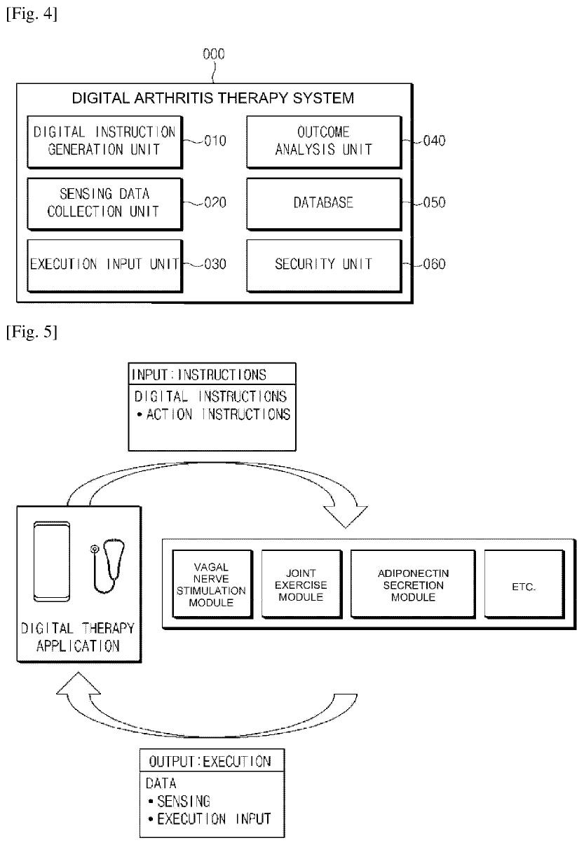

FIG. 4 is a block diagram showing a configuration of the digital apparatus for preventing and/or ameliorating arthritis according to one embodiment of the present disclosure. Referring to FIG. 4, a digital system 000 for preventing and/or ameliorating arthritis according to one embodiment of the present disclosure may include a digital instruction generation unit 010, a sensing data collection unit 020, an execution input unit 030, an outcome analysis unit 040, a database 050, and a security unit 060.

Based on the mechanism of action in and the therapeutic hypothesis and digital therapeutic hypothesis for arthritis, a doctor (a second user) may prescribe digital therapeutics, which are realized in a digital apparatus and an application for preventing and/or ameliorating arthritis, for the corresponding patient. In this case, the digital instruction generation unit 010 is a device configured to provide a prescription of the digital therapeutics to a patient as a specific behavioral instruction that the patient may execute based on the interaction between the biochemical factors for arthritis and the patient's behaviors. For example, the biochemical factors may include adiponectin, Oncostatin M, IL-6, TNF-α, biochemical factor associated with inflammation or anti-inflammation, and the like, but the present disclosure is not limited thereto. For example, all types of biochemical factors that may cause arthritis may be considered.

The digital instruction generation unit 010 may generate digital instructions based on the inputs from the doctor. In this case, the digital instruction generation unit 010 may generate digital instructions based on information collected by the doctor when diagnosing a patient. Also, the digital instruction generation unit 010 may generate digital instructions based on the information received from the patient. For example, the information received from the patient may include the patient's basal factors, medical information, and digital therapeutics literacy. In this case, the basal factors may include amount of the patient's activity, heart rates, sleep, meals (nutrition and calories), and the like. The medical information may include the patient's electronic medical record (EMR), family history, genetic vulnerability, genetic susceptibility, and the like. The digital therapeutics literacy may include the patient's accessibility and an acceptance posture to the digital therapy instructions and the apparatus, and the like.

The digital instruction generation unit 010 may reflect the mechanism of action in and the therapeutic hypothesis for arthritis in order to utilize imaginary parameters and generate a digital module. In this case, the imaginary parameters may be deduced in term of the patient's behaviors.

The digital instruction generation unit 010 generates digital instructions particularly designed to allow a patient to have a therapeutic effect, and provides the instructions to the patient. For example, the digital instruction generation unit 010 may generate specific digital instructions in each of digital therapeutic modules.

The sensing data collection unit 020 and the execution input unit 030 may collect the patient's execution outcomes of the digital instructions provided at the digital instruction generation unit 010. Specifically, the sensing data collection unit 020 configured to sense the patient's adherence to the digital instructions and the execution input unit 030 configured to allow a patient to directly input the execution outcomes of the digital instructions are included, and thus serve to output the patient's execution outcomes of the digital instructions.

The outcome analysis unit 040 may collect the patient's behavior adherence or participation in predetermined periods and report the patient's behavior adherence or participation to external systems. Therefore, a doctor may continue to monitor an execution course of the digital instructions through the application even when a patient does not directly visit a hospital.

The database 050 may store the mechanism of action in arthritis, the therapeutic hypothesis for arthritis, the digital instructions provided to the user, and the user's execution outcome data. FIG. 4 shows that the database 050 is included in the digital apparatus 000 for preventing and/or ameliorating arthritis. However, the database 050 may be provided in an external server.

Meanwhile, a series of loops including inputting the digital instructions at the digital instruction generation unit 010, outputting the patient's execution outcomes of the digital instructions at the sensing data collection unit 020/execution input unit 030, and evaluating the execution outcomes at the outcome analysis unit 040 may be repeatedly executed several times. In this case, the digital instruction generation unit 010 may generate patient-customized digital instructions for this cycle by reflecting the patient's digital instructions provided in the previous cycle and output values, and the evaluation.

As described above, according to the digital therapy apparatus for inhibiting progression of and preventing and/or ameliorating arthritis according to the present disclosure, the arthritis therapy whose reliability may be ensured is possible by deducing the mechanism of action in arthritis and the therapeutic hypothesis and digital therapeutic hypothesis for arthritis in consideration of the biochemical factors for arthritis, presenting digital instructions for preventing and/or ameliorating arthritis based on the mechanism of action and the therapeutic hypotheses, and collecting and analyzing execution of specific instructions.

FIG. 5 is a diagram showing input and output loops of the digital application for preventing and/or ameliorating arthritis according to one embodiment of the present disclosure.

Referring to FIG. 5, the digital application for preventing and/or ameliorating arthritis according to one embodiment of the present disclosure may input the corresponding digital prescription for a patient in the form of instructions, and may output execution outcomes of the corresponding digital instructions.

The digital instructions provided to the patient may include specific action instructions for behaviors, and the like. As shown in FIG. 5, the digital instructions may include vagal nerve stimulation, joint exercise, adiponectin secretion, and the like. However, the digital instructions are given by way of illustration only, and are not intended to be limiting to the digital instruction according to the present disclosure.

The patient's execution outcomes of the digital instructions consist of 1) log-in/log-out information for instructions and execution, 2) adherence information sensed as passive data such as joint exercise, heart rates associated with the stress, a change in oxygen saturation, and the like, and 3) directly input information on the patient's execution outcomes.

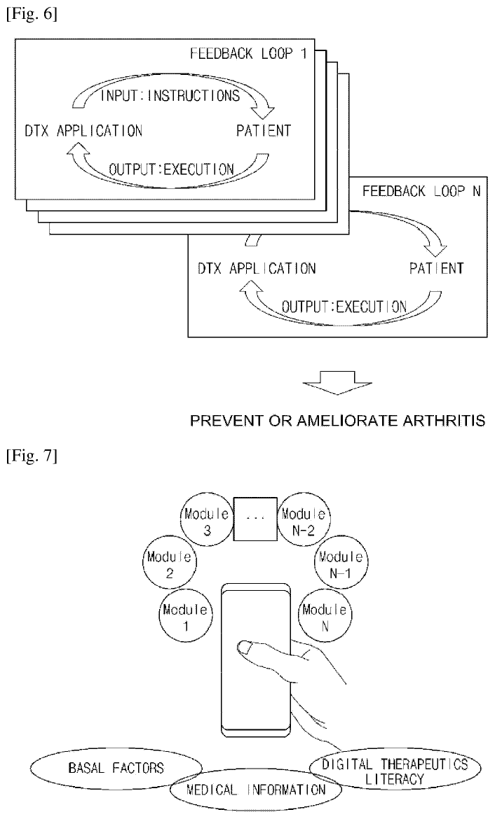

FIG. 6 is a diagram showing a feedback loop for the digital apparatus and the application for preventing and/or ameliorating arthritis according to one embodiment of the present disclosure. Referring to FIG. 6, the inhibition of the progression of and the treatment of arthritis are shown to be achieved by repeatedly executing the aforementioned single feedback loop of FIG. 5 several times to regulate the biochemical factors.

Inhibitory and therapeutic effects on progression of the arthritis may be more effectively achieved by gradual improvement of an instruction-execution cycle in the feedback loop, compared to the simply repeated instruction-execution cycle during the corresponding course of therapy.

For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the digital apparatus and the application for preventing and/or ameliorating arthritis according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary.

As described above, in the digital apparatus and the application for preventing and/or ameliorating arthritis according to one embodiment of the present disclosure, it is possible to optimize the patient-customized digital instructions suitable for the patient through the rapid feedback loop.

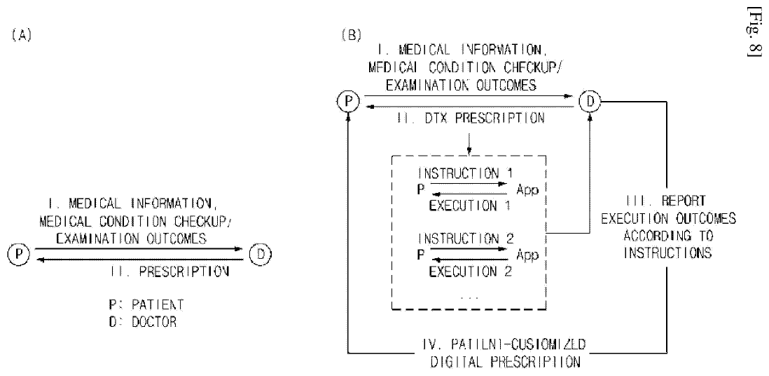

FIG. 7 is a diagram showing a background factors supporting the digital apparatus and the application for preventing and/or ameliorating arthritis according to one embodiment of the present disclosure

Referring to FIG. 7, the background factors may be considered together in the design of the modules in the digital apparatus and the application for preventing and/or ameliorating arthritis according to one embodiment of the present disclosure.

In this case, the background factors are elements necessary for correction of clinical trial outcomes during verification of the clinical effectiveness of digital arthritis therapy according to the present disclosure. Specifically, in the background factors shown in FIG. 7, the basal factors may include activity, heart rates, sleep, meals (nutrition and calories), and the like, the medical information may include EMR, family history, genetic vulnerability, and susceptibility, and the like, which have been written when a patient visited a hospital, and the digital therapeutics literacy may include the patient's accessibility to the digital therapy instructions and the apparatus, and an acceptance posture.

FIG. 8 is a diagram showing a method of assigning a patient-customized digital prescription using the digital apparatus and the application for preventing and/or ameliorating arthritis according to one embodiment of the present disclosure.

FIG. 8(A) show a prescription procedure for routine medical condition checkup of a patient by a doctor, and FIG. 8(B) show a method of allowing a doctor to assign a patient-customized digital prescription based on the analysis of a plurality of digital instructions and execution outcomes of the digital instructions.

In this way, when the digital apparatus and the application for preventing and/or ameliorating arthritis according to one embodiment of the present disclosure are used, the doctor may check the patient's instructions and execution outcomes for a given period and adjust the types of modules for preventing and/or ameliorating arthritis, and the instructions for each module in a patient-customized manner, as shown in FIG. 8(B).

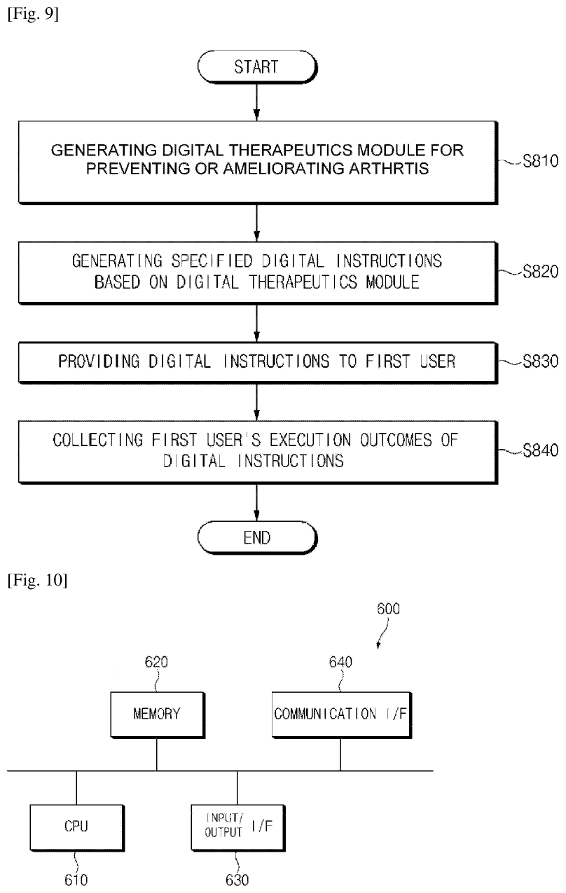

FIG. 9 is a flowchart illustrating operations in the digital application for preventing and/or ameliorating arthritis according to one embodiment of the present disclosure.

Referring to FIG. 9, the digital application for preventing and/or ameliorating arthritis according to one embodiment of the present disclosure may first generate a digital therapeutics module for preventing and/or ameliorating arthritis based on the mechanism of action in and the therapeutic hypothesis for arthritis (S810). In this case, in S810, the digital therapeutics module may be generated based on the biochemical factors (for example, adiponectin, Oncostatin M, IL-6, TNF-α, biochemical factor associated with inflammation or anti-inflammation, and the like) for arthritis.

Meanwhile, in S810, the digital therapeutics module may be generated based on the inputs from the doctor. In this case, a digital therapeutics module may be generated based on the information collected by the doctor when diagnosing a patient, and the prescription outcomes recorded based on the information. Also, in S810, the digital therapeutics module may be generated based on the information (for example, basal factors, medical information, digital therapeutics literacy, etc.) received from the patient.

Next, in S820, specified digital instructions may be generated based on the digital therapeutics module. S820 may generate a digital therapeutics module by applying imaginary parameters about the patient's environments, behaviors, emotions, and cognition to the mechanism of action in and the therapeutic hypothesis for arthritis.

In this case, the digital instructions may be generated for at least one of vagal nerve stimulation, joint exercise, and adiponectin secretion modules. Then, the digital instructions may be provided to a patient (S830). In this case, the digital instructions may be provided in the form of digital instructions which are associated with behaviors and in which the patient's instruction adherence such as joint exercise may be monitored using a sensor, or provided in the form of digital instructions in which a patient is allowed to directly input the execution outcomes.

After the patient executes the presented digital instructions, the patient's execution outcomes of the digital instructions may be collected (S840). In S840, the execution outcomes of the digital instructions may be collected by monitoring the patient's adherence to the digital instructions as described above, or allowing the patient to input the execution outcomes of the digital instructions.

Meanwhile, the digital application for preventing and/or ameliorating arthritis according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for preventing and/or ameliorating arthritis according to one embodiment of the present disclosure, the reliability of the inhibition of progression of and treatment of arthritis may be ensured by deducing the mechanism of action in and the therapeutic hypothesis for arthritis in consideration of the biochemical factors for arthritis, presenting the digital instructions to a patient based on the mechanism of action in and the therapeutic hypothesis for arthritis, and collecting and analyzing the outcomes of the digital instructions.

Although the digital apparatus and the application for preventing and/or ameliorating arthritis according to one embodiment of the present disclosure have been described in terms of arthritis therapy, the present disclosure is not limited thereto. For the other diseases other than the arthritis, the digital therapy may be executed substantially in the same manner as described above.

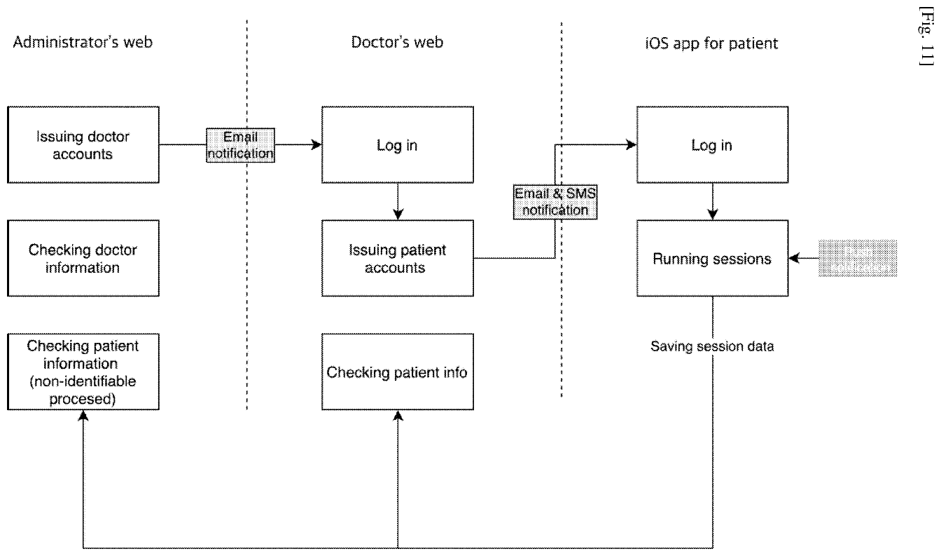

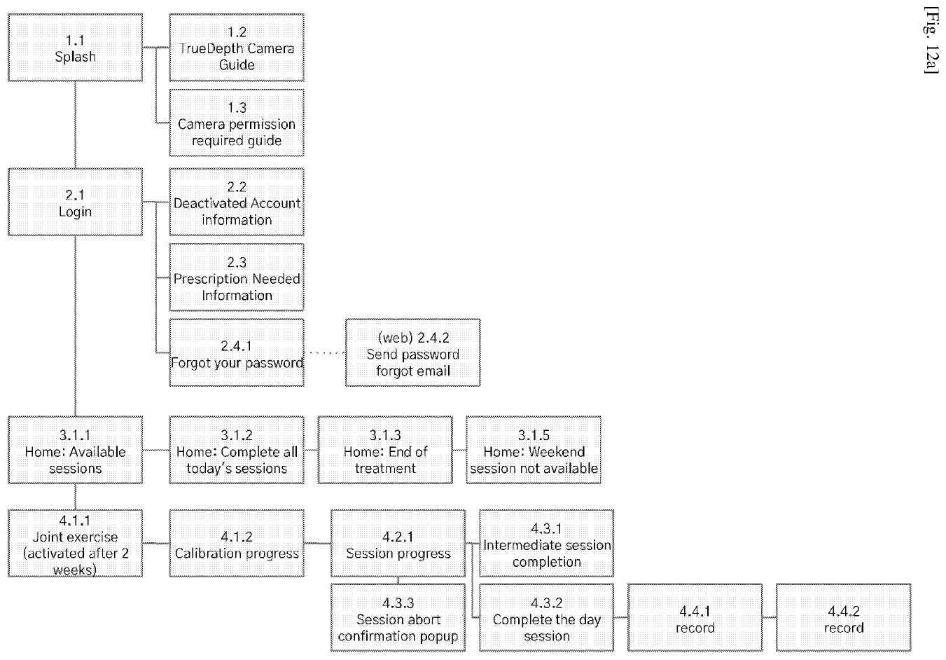

FIG. 10 is a diagram showing a hardware configuration of the digital apparatus for preventing and/or ameliorating arthritis according to one embodiment of the present disclosure.

Referring to FIG. 10, hardware 600 of the digital apparatus for preventing and/or ameliorating arthritis according to one embodiment of the present disclosure may include a CPU 610, a memory 620, an input/output I/F 630, and a communication I/F 640.

The CPU 610 may be a processor configured to execute a digital program for preventing and/or ameliorating arthritis stored in the memory 620, process various data for digitally treating arthritis and execute functions associated with the digital arthritis therapy. That is, the CPU 610 may act to execute functions for each of the configurations shown in FIG. 4 by executing the digital program for preventing and/or ameliorating arthritis stored in the memory 620.

The memory 620 may have a digital program for preventing and/or ameliorating arthritis stored therein. Also, the memory 620 may include the data used for the digital arthritis therapy included in the aforementioned database 050, for example, the patient's digital instructions and instruction execution outcomes, the patient's medical information, and the like.

A plurality of such memories 620 may be provided, when necessary. The memory 620 may be a volatile memory or a non-volatile memory. When the memory 620 is a volatile memory, RAM, DRAM, SRAM, and the like may be used as the memory 620. When the memory 620 is a non-volatile memory, ROM, PROM, EAROM, EPROM, EEPROM, a flash memory, and the like may be used as the memory 620. Examples of the memories 620 as listed above are given by way of illustration only, and are not intended to limit the present disclosure.

The input/output I/F 630 may provide an interface in which input apparatuses (not shown) such as a keyboard, a mouse, a touch panel, and the like, and output apparatuses such as a display (not shown), and the like may transmit and receive data (e.g., wirelessly or by hardline) to the CPU 610.

The communication I/F 640 is configured to transmit and receive various types of data to/from a server, and may be one of various apparatuses capable of supporting wire or wireless communication. For example, the types of data on the aforementioned digital behavior-based therapy may be received from a separately available external server through the communication I/F 640.

As described above, the computer program according to one embodiment of the present disclosure may be recorded in the memory 620 and processed at the CPU 610, for example, so that the computer program may be realized as a module configured to execute each of functional blocks shown in FIG. 4.

According to the digital apparatus and the application for treating, ameliorating, or preventing arthritis according to the present disclosure, a reliable digital apparatus and application capable of inhibiting progression of and preventing and/or ameliorating arthritis may be provided by deducing a mechanism of action in arthritis and a therapeutic hypothesis and a digital therapeutic hypothesis for arthritis in consideration of biochemical factors for progression of arthritis, presenting digital instructions to a patient, and collecting and analyzing execution outcomes of the digital instructions.

In certain aspects, the present disclosure provides a method of treating, ameliorating, or preventing arthritis in a joint of a subject in need thereof. In some embodiments, the method comprises providing, by an electronic device to the subject, one or more first modules selected from the group consisting of a vagal nerve stimulation module, a joint exercise module, and an adiponectin secretion module, each of the one or more first modules comprising one or more first instructions for the subject to follow. In some embodiments, the method comprises sensing, using a sensor of the electronic device, adherence by the subject to the first instructions of the one or more first modules, wherein the electronic device transmits adherence information, based on the adherence, to a server, and receives one or more second instructions from the server based on the adherence information. In some embodiments, the method comprises providing, by the electronic device to the subject, one or more second modules selected from the group consisting of an adiponectin secretion module, a vagal nerve stimulation module, and a joint exercise module, each of the one or more first modules comprising one or more second instructions.

In certain aspects, the present disclosure provides a system for treating, ameliorating, or preventing arthritis in a joint of a subject in need thereof. In some embodiments, the system comprises a digital apparatus configured to execute a digital application comprising one or more first modules for preventing or ameliorating arthritis in a joint of the subject. In some embodiments, the digital apparatus comprises a sensor for sensing adherence by the subject to a first set of instructions of the one or more first modules. In some embodiments, the system comprises a healthcare provider portal configured to provide one or more options to a healthcare provider to perform one or more tasks to prescribe treatment for preventing or ameliorating arthritis in a joint of the subject based on information received from the digital application. In some embodiments, the system comprises an administrative portal configured to provide one or more options to an administrator of the system to perform one or more tasks to manage access to the system by the healthcare provider.

In certain aspects, the present disclosure provides a computing system for preventing or ameliorating arthritis in a joint of a subject in need thereof. In some embodiments, the computing system comprises a display configured to provide, to the subject, one or more first modules selected from the group consisting of a vagal nerve stimulation module, a joint exercise module, and an adiponectin secretion module, each of the one or more first modules comprising one or more first instructions for the subject to follow. In some embodiments, the computing system comprises a sensor configured to sense adherence by the subject to the instructions of the one or more first modules. In some embodiments, the computing system comprises a transmitter configured to transmit adherence information, based on the adherence, to a server. In some embodiments, the computing system comprises a receiver configured to receive, from the server, one or more second instructions based on the adherence information.

In some aspects, the present disclosure provides a non-transitory computer readable medium having stored thereon software instructions for preventing or ameliorating arthritis in a joint of a subject in need thereof that, when executed by a processor, cause the processor to display, by an electronic device to the subject, one or more first modules selected from the group consisting of one or more first modules selected from the group consisting of a vagal nerve stimulation module, a joint exercise module, and an adiponectin secretion module, each of the one or more first modules comprising one or more first instructions for the subject to follow. In some aspects, the present disclosure provides a non-transitory computer readable medium having stored thereon software instructions for preventing or ameliorating arthritis in a joint of a subject in need thereof that, when executed by a processor, cause the processor to sense, by a sensor in the electronic device, adherence by the subject to the instructions of the one or more first modules. In some aspects, the present disclosure provides a non-transitory computer readable medium having stored thereon software instructions for preventing or ameliorating arthritis in a joint of a subject in need thereof that, when executed by a processor, cause the processor to transmit, by the electronic device, adherence information, based on the adherence, to a server. In some aspects, the present disclosure provides a non-transitory computer readable medium having stored thereon software instructions for preventing or ameliorating arthritis in a joint of a subject in need thereof that, when executed by a processor, cause the processor to receive, from the server, one or more second instructions based on the adherence information.

In some embodiments, the applications, methods and systems of the present disclosure are used to prevent or ameliorate arthritis. Arthritis can generally be characterized as a joint disorder or condition that involves inflammation, causing acute or chronic pain. A variety of types and subtypes of arthritis of various etiologies and causes, either known or unknown, including, but not limited to, rheumatoid arthritis, osteoarthritis, infectious arthritis, psoriatic arthritis, gouty arthritis, and lupus-related arthritis. Non-limiting examples of arthritis include osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

In certain embodiments, applications, methods or systems of the present disclosure are used to prevent and/or ameliorate rheumatoid arthritis in a subject. Rheumatoid arthritis is an autoimmune disorder that mainly involves the joints, which become swollen and painful. The disease may also affect other parts of the body, leading for example to a reduction in the number of red blood cells, the appearance of inflammation around the heart and lungs, fever and a general sense of fatigue. It is believed that this disease is due to a combination of genetic and environmental factors. The main mechanism involves the activation of the immune system against the joints, which leads to inflammation and thickening of the articular capsule, also involving the bone and cartilage. The initial site of the disease is the synovial membrane, where there is an infiltration of cells of the immune system. More specifically, rheumatoid arthritis is generally characterized by the succession of three phases: an initial phase due to non-specific inflammation, an amplification phase involving the activation of T cells, and finally a final phase of chronic inflammation due to the release of pro-inflammatory cytokines, such as interleukin-2, Y-interferon, tumor necrosis factor (TNF) and interleukin-6.

In certain embodiments, applications, methods or systems of the present disclosure are used to prevent and/or ameliorate osteoarthritis in a subject. Osteoarthritis (osteoarthrosis or arthrosis) is a degenerative disease of the joints (e.g., hips, hands and knees). It is well known that the synovia, bone and cartilage are the tissues most involved in the pathological mechanisms of osteoarthritis. The cause of the disease is likely to be related to changes in the homeostasis of the joint cartilage and bone that lead to the increase of destructive processes. In osteoarthritis there is a change in the structure of the bone and a degeneration of the joint cartilage. The subchondral bone becomes rigid, less capable of absorbing impact loads, which leads to greater stress on the cartilage. In fact, the main characteristics of the disease include a progressive loss of cartilage tissue, hypertrophic bone modifications and formation of osteophytes that grow at the edges of the bones involved. With reference to cartilage, it is known that chondrocytes are the cells responsible for balancing the processes of synthesis and destruction of the matrix, capable of regulating cytokines and growth factors. In degenerative osteoarticular processes, this balance is usually compromised. In patients with osteoarthritis, chondrocytes produce high levels of inflammatory cytokines such as interleukin lβ (IL-lβ) and tumor necrosis factor (TNF), which in turn reduce collagen synthesis and increase concentrations of catabolic mediators such as metalloproteases. At the same time, other pro-inflammatory substances such as interleukin-8 (IL-8) and interleukin-6 (IL-6), prostaglandin E2 and nitric oxide are released. The increase of oxidizing agents such as nitric oxide determines the apoptosis of chondrocytes and thus the degeneration of the matrix.

In certain embodiments, applications, methods or systems of the present disclosure are used to prevent and/or ameliorate infectious arthritis in a subject. Infectious arthritis general refers to joint inflammations derived from bacterial, mycosic or viral infections. Acute infectious arthritis is generally of bacterial or viral origin. With regard to bacterial arthritis, the bacteria involved are gonococchi, staphylococci, streptococci, pneumococci, Haemophilus, Spirochetes. Non-gonococcal arthritis is usually caused by Staphylococcus aureus, Spirochetes, streptococci or other grambacteria. With respect to acute viral arthritis, the chief identified causes are B19 parvoviruses, flaviviruses, Hepatitis B and C viruses, the rubella and measles viruses, togaviruses (including alphaviruses). Other pathologies are also associated with arthralgia and arthritis. These are chicken pox, mumps, adenovirus illnesses, Coxsackie A9, B2, B3, B4 and B6 viruses, and Epstein-Barr mononucleosis. In all these situations, the infection leads to an inflammatory reaction at the joints. The clinical signs of infectious arthritis are joint pain and tumefaction at the site of inflammation. This may affect several joints. These signs are usually of sudden onset, accompanied by fever and shivering. Treatments currently available have recourse to non-steroid anti-inflammatory drugs (NSAIDs) in particular to relieve pain, swelling and stiffness caused by infectious arthritis, but they do not contribute towards preventing joint lesions and their consequences. In stronger doses NSAIDs also help to relieve inflammation. For infectious arthritis of bacterial origin antibiotics are used, but for arthritis of viral origin generally no medication is prescribed so as to treat the cause of the pathology.

In certain embodiments, applications, methods or systems of the present disclosure are used to prevent and/or ameliorate psoriatic arthritis in a subject. Psoriatic arthritis is an inflammatory condition that affects the joints of both children and adults with psoriasis. Psoriasis is a skin condition that causes patches of thick, red skin to form on certain areas of your body. Psoriatic arthritis may affect one joint or many. Tumor necrosis factor has been implicated in the pathophysiology of psoriatic arthritis. Signs and symptoms of psoriatic arthritis include pain in affected joints, swollen joints, and joints that are warm to the touch. Psoriatic arthritis can be debilitating and painful, making it difficult for those affected to perform even daily routines. Despite medications, psoriatic arthritis can also cause erosion in joints of patients having psoriatic arthritis. No cure exists for psoriatic arthritis. Generally, treatment includes trying to control inflammation in affected joints in order to prevent joint pain and disability. Medications commonly used to treat psoriatic arthritis include nonsteroidal anti-inflammatory drugs (NSAIDs), corticosteroids, and disease-modifying anti-rheumatic drugs (DMARDs).

In certain embodiments, applications, methods or systems of the present disclosure are used to prevent and/or ameliorate gouty arthritis in a subject. Gout is caused by a defect in metabolism that results in an overproduction of uric acid or a reduced ability of the kidney to eliminate uric acid. The exact cause of the metabolic defect is unknown. In acute gouty arthritis, symptoms develop suddenly and usually involve only one or a few joints. The joint appears infected with signs of warmth, redness, and tenderness. The attacks of painful joints may subside in several days, but may recur at irregular intervals. Subsequent attacks usually have a longer duration. Some people may progress to chronic gouty arthritis. The symptoms of gout include joint pain that affects one or more joints (e.g. hip pain, knee pain, ankle pain, foot pain, shoulder pain, elbow pain, wrist pain, hand pain, or pain in other joints). The great toe, knee, or ankle joints are most often affected. Other symptoms include joint swelling of the affected joints, stiffness of the joint, warm and red joints, fever, skin lumps. There is no known cure for gout or gouty arthritis. The goals of treatment are mainly to stop the pain and inflammation associated with the initial attack, and to prevent future attacks.

In certain embodiments, applications, methods or systems of the present disclosure are used to prevent and/or ameliorate lupus-related arthritis in a subject. Lupus-related arthritis causes pain, stiffness, swelling, tenderness, and warmth in the joints in a waxing and waning pattern. The joints most often affected are the ones farthest from the middle of the body, such as fingers, wrists, elbows, knees, ankles, and toes. General stiffness when upon waking up in the morning, which gradually improves as the day goes on, is a key feature of lupus-related arthritis. In lupus-related arthritis, several joints are usually involved, and the inflammation will affect similar joints on both sides of the body. All major and minor joints may be affected. Compared to rheumatoid arthritis, however, lupus-related arthritis is less disabling and less likely to cause destruction of the joints. Fewer than 10 percent of people with lupus-related arthritis will develop deformities of their hands and feet associated with weakening of cartilage and bone. There is no definitive treatment or cure for lupus-related arthritis. The principal goals of therapy are to relieve symptoms and improve function. According to the Lupus Foundation of America, current treatment for lupus-related arthritis has five basic goals: reduce inflammation, suppress immune system, prevent and treat flare ups of the condition, control symptoms and limit any damage to the body and organs. Nonsteroidal anti-inflammatories (NSAIDs), corticosteroids, antimalarial drugs and a variety of immunosuppressive medications are the standard of care for patients with lupus-related arthritis.

A module comprising physical exercise can include, for example, 20 minutes of high intensity exercise and 40 minutes of rest or low intensity exercise in a patient-bearable situation (for example, a module that mimics a low mountain climb, walk, etc.). This module can be repeated daily, and the effects observed after 1 week, 2 weeks, 3 weeks, 1 month, 2 months, 3 months, 4 months, 5 months, 6 months, 9 months, or 1 year.

In certain embodiments, the one or more first modules comprise the vagal nerve stimulation module, and the vagal nerve stimulation module comprises one or more first instructions to reduce inflammation in the subject. In certain embodiments, the one or more first modules comprise the vagal nerve stimulation module, and the vagal nerve stimulation module comprises at least one instructions selected from the group consisting of sense stimulation instructions for sight, sound, touch, taste, and smell. In certain embodiments, the vagal nerve stimulation module comprises one or more sense stimulation instructions for sight. In certain embodiments, the one or more sense stimulation instructions for sight include one or more instructions to view one or more figures to stimulate autonomic nervous system. In certain embodiments, the vagal nerve stimulation module comprises one or more sense stimulation instructions for sound. In certain embodiments, the one or more sense stimulation instructions for sound include one or more instructions to hear one or more sounds to cause honor or relaxation. In certain embodiments, the vagal nerve stimulation module comprises one or more sense stimulation instructions for touch. In certain embodiments, the one or more sense stimulation instructions for touch include one or more instructions for abdominal breathing, controlling rate of breathing, cold massage, coughing, and skin massage. In certain embodiments, the vagal nerve stimulation module comprises one or more sense stimulation instructions for touch. In certain embodiments, the one or more sense stimulation instructions for touch include one or more instructions for abdominal breathing. In certain embodiments, the vagal nerve stimulation module comprises one or more sense stimulation instructions for taste. In certain embodiments, the one or more sense stimulation instructions for taste include one or more instructions to eat food to stimulate digestive glands in the subject. In certain embodiments, the vagal nerve stimulation module comprises one or more sense stimulation instructions for smell. In certain embodiments, the one or more sense stimulation instructions for smell include one or more instructions to stimulate digestive glands and/or to relax.

In some embodiments, the one or more first modules comprise an adiponectin secretion module, comprising one or more instructions for the subject to follow that increase adiponectin secretion in the subject. In some embodiments, the adiponectin secretion module comprises one or more of an aerobic exercise module and a relaxation module.

In certain embodiments, the aerobic exercise module comprises one or more first instructions to increase adiponectin secretion in the subject. In certain embodiments, the one or more first instructions comprise one or more instructions for walking, biking, aerobic dance and/or swimming.

In certain embodiments, the relaxation module comprises one or more first instructions to increase adiponectin secretion in the subject. In certain embodiments, the one or more first instructions to increase adiponectin secretion in the subject comprise one or more meditation instructions. In certain embodiments, the one or more first instructions to increase adiponectin secretion in the subject comprise one or more sound instructions to hear relaxing sound. One mechanism by which adiponectin can be increased is vagal nerve stimulation, which produces an anti-inflammatory effect and proteolysis block. In some embodiments, the relaxation module comprises meditation. In some embodiments, the relaxation module comprises deep breathing. In some embodiments, the relaxation module enables abdominal nerve stimulation. In some embodiments, the relaxation module is performed in an atmosphere or environment that is relaxing to the patient. In some embodiments, the relaxation module comprises listening to music. This module can be repeated daily, and the effects observed after 1 week, 2 weeks, 3 weeks, 1 month, 2 months, 3 months, 4 months, 5 months, 6 months, 9 months, or 1 year.

In certain embodiments, cold massage can be used to reduce pain and or inflammation in an arthritic joint of the subject. However, in other embodiments, the one or more first modules do not comprise one or more first instructions for a cold massage.

In certain embodiments, the one or more first modules comprise a joint exercise module. The joint exercise module induces normalization of the balance between (i) sympathetic innervation on the synovial membrane, (ii) sensory innervation on the synovial membrane, and (iii) Oncostatin M production. In some embodiments, the joint exercise module comprises one or more first instructions to reduce inflammation in the joint of the subject. In some embodiments, a joint exercise module comprises bending, twisting, rotating, and/or stretching a joint of the subject in a predetermined manner. In other embodiments, a joint exercise can comprise walking or running at a predetermined speed. A joint can refer to any joint of the body, including but not limited to, a shoulder joint, an elbow joint, a sacroiliac joint, a hip joint, a knee joint, a joint of the wrist or hand, a joint of the ankle or foot, a neck joint, or an intervertebral joint.

In some embodiments, a joint exercise comprises bending a joint at a predetermined angle. For example, a subject can be instructed to bend their fingers at a predetermined angle. Using a sensor on the electronic device, the device can detect the angle at which the subject is bending the joint, compare the angle to the predetermined angle at which the subject should bend the joint, and provide feedback to the subject (e.g., to bend the joint less or more). The predetermined angle can be measured, for example, by degrees (e.g, 90 degrees). It will be understood that a person of skill could choose any predetermined angle for the subject to bend the joint.

In some embodiments, a joint exercise comprises bending a joint at a predetermined angle for a predetermined period of time. The predetermined period of time can be, for example, 5 seconds, 10 seconds, 30 seconds, 45 seconds, 1 minute, 2 minutes, 3 minutes, 4 minutes, 5 minutes, or greater than 5 minutes. In some embodiments, a joint exercise comprises repeatedly bending a joint at a predetermined angle for a predetermined period of time. In some embodiments, the joint exercise is repeated 1 time, 2 times, 3 times, 4 times, 5 times, 6 times, 7 times, 8 times, 9 times, 10 times, 15 times, 20 times, 25 times, 50 times, or greater than 50 times.

A session may comprise any number of digital therapeutic modules. In some embodiments, a session may comprise two or more digital therapeutic modules. In some embodiments, a session may comprise 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more digital therapeutic modules. A session may comprise any number of digital therapeutic modules, and the digital therapeutic modules may be independently selected from a vagal nerve stimulation module, a joint exercise module, and an adiponectin secretion module. In some embodiments, a session may consist of 3 digital therapeutic modules, and the digital therapeutic modules comprise a vagal nerve stimulation module, a joint exercise module, and an adiponectin secretion module. A person of skill in the art will appreciate that there are a vast number of combinations for the number and type(s) of digital therapeutic modules that may go into a particular session. For each combination, a person of skill in the art will appreciate that the various digital therapeutic modules can be performed in multiple different orders. A session can be repeated as frequently or as infrequently as needed. In some embodiments, a session can be repeated 5 times per day, 4 times per day, 3 times per day, 2 times per day, daily, every 2 days, every 3 days, every 4 days, every 5 days, every 6 days, every 7 days, every 2 weeks, every 3 weeks, or every 4 weeks.

In some embodiments, the external reviewer comprises a health professional (e.g., a healthcare provider or doctor). In some embodiments, the external reviewer comprises an artificial intelligence (AI). The term “artificial intelligence” can refer to intelligence exhibited by machines. In computer science, an ideal “intelligent” machine is a flexible rational agent that perceives its environment and takes actions that maximize its chance of success at some goal (e.g., preventing and/or ameliorating arthritis in a patient). Colloquially, the term “artificial intelligence” is applied when a machine mimics “cognitive” functions that humans associate with other human minds, such as “learning” and “problem solving. The term artificial intelligence may refer to an algorithm that may apply learning skills on multiple types of information (such as physiological information, additional information and person's medical history).

In some embodiments, the digital apparatus comprises a sensor, and the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer. In some embodiments, the sensor comprises a touch sensor, and the subject provides the adherence information to the electronic device using the touch sensor.

FIG. 11 depicts a flow chart illustrating a system for preventing and/or ameliorating arthritis, the system comprising an administrative portal (e.g., Administrator's web), a healthcare provider portal (e.g., Doctor's web) and a digital apparatus configured to execute a digital application (e.g., an application or ‘app’) for preventing and/or ameliorating arthritis in a subject. Among other things, the Administrator's portal allows an administrator to issue doctor accounts, review doctor information, and review de-identified patient information. Among other things, the Healthcare Provider's portal allows a healthcare provider (e.g., a doctor) to issue patient accounts, and review patient information (e.g., age, prescription information, and status for having completed one or more digital therapeutic modules or sessions). Among other things, the digital application allows a patient access to complete one or more digital therapeutic modules or sessions.

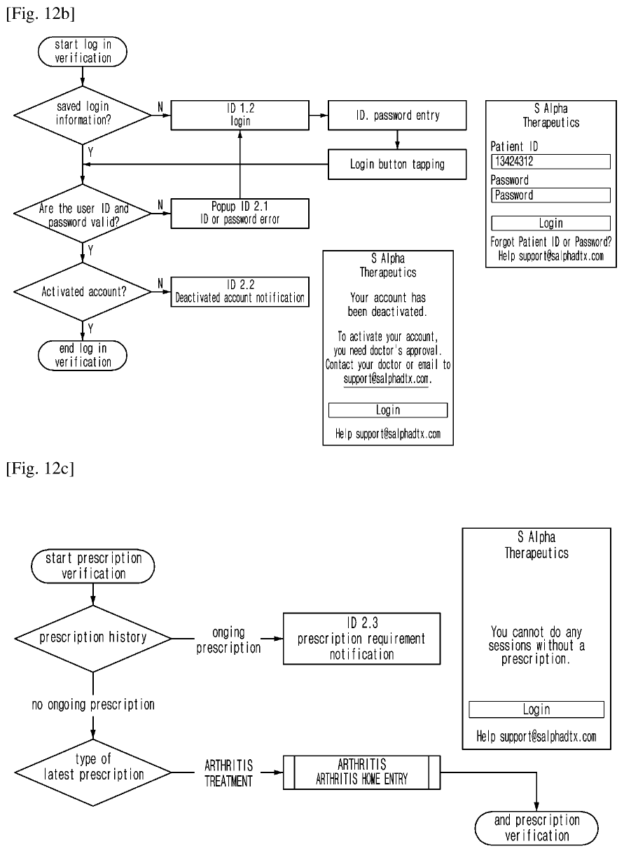

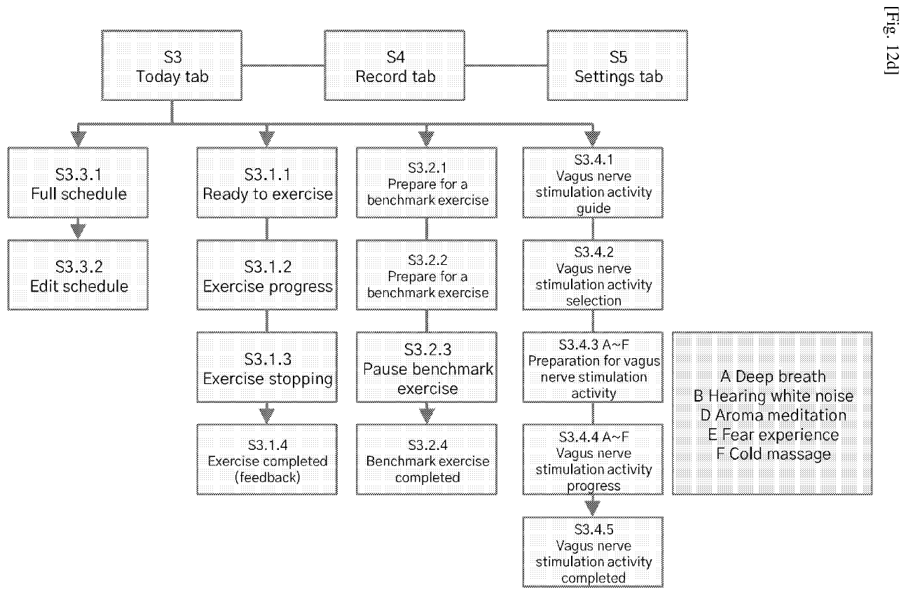

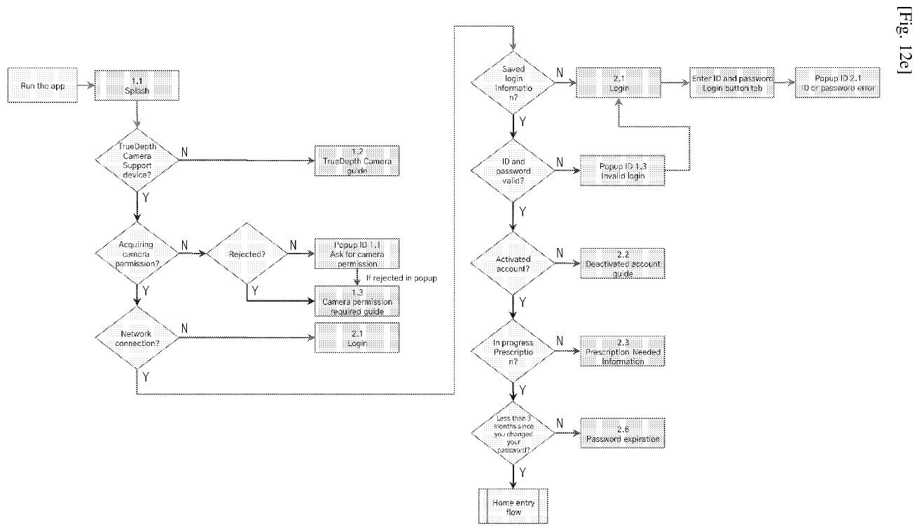

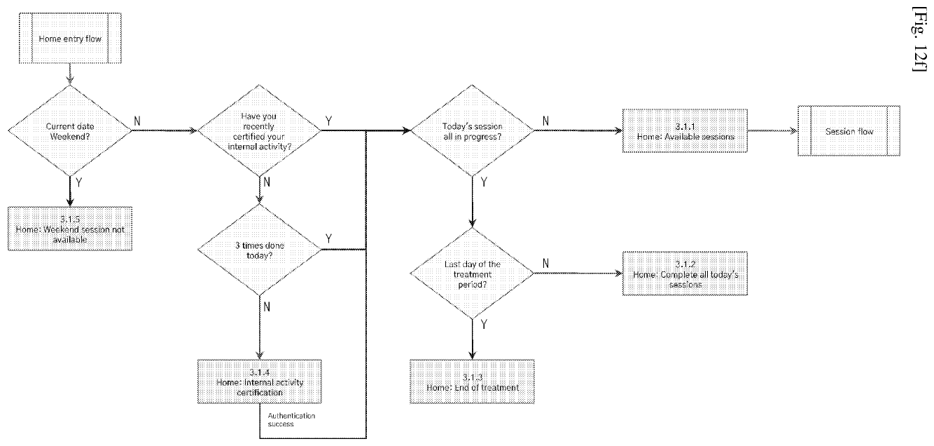

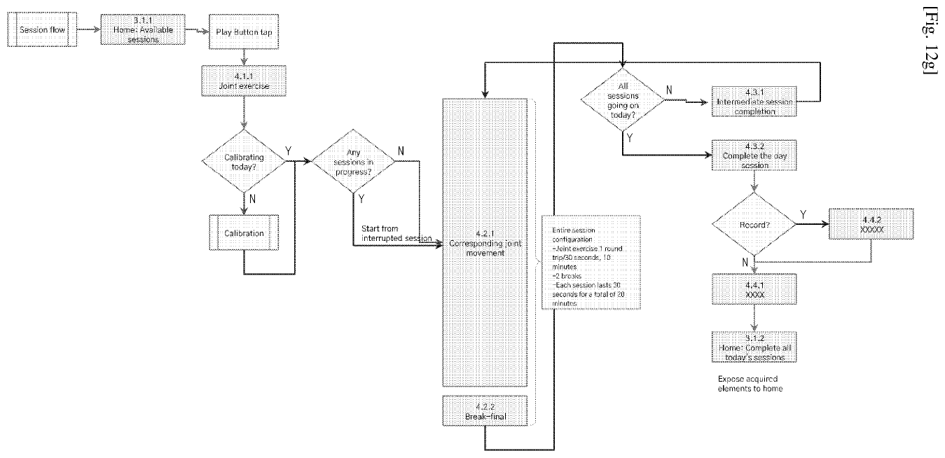

FIG. 12B depicts a flow chart illustrating an execution flow for login verification during a splash process at the starting of the digital application. Similarly, FIG. 12C depicts a flow chart illustrating an execution flow for prescription verification during a splash process at the starting of the digital application. The prescription verification process may comprise, for example, determining if the treatment period has expired, determining whether the subject has been recently (e.g., within the last hour) performed a joint exercise, determining if, based on the prescription, the subject's sessions for the day have been completed (e.g., the subject is compliant with the prescription). In such instances, the digital apparatus may notify the subject that there are no sessions available to be completed. FIG. 12D depicts a flow chart illustrating exemplary execution flow of a schedule of modules to be performed, and performance of the scheduled module. FIG. 12E depicts a flow chart of the application execution flow from starting the application through entry to the home screen. FIG. 12F depicts a flow chart of the application execution flow from the home screen through the start of providing one or more modules to the subject. FIG. 12G depicts a flow chart of the application execution flow from the start of providing one or more modules to the subject through completion of the scheduled modules.

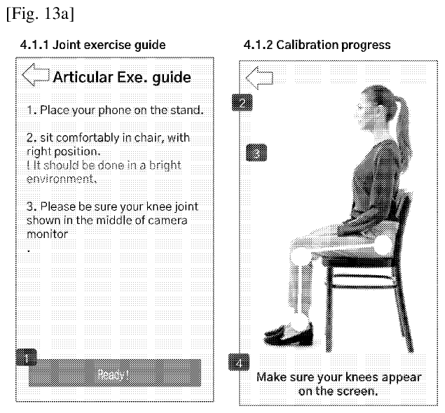

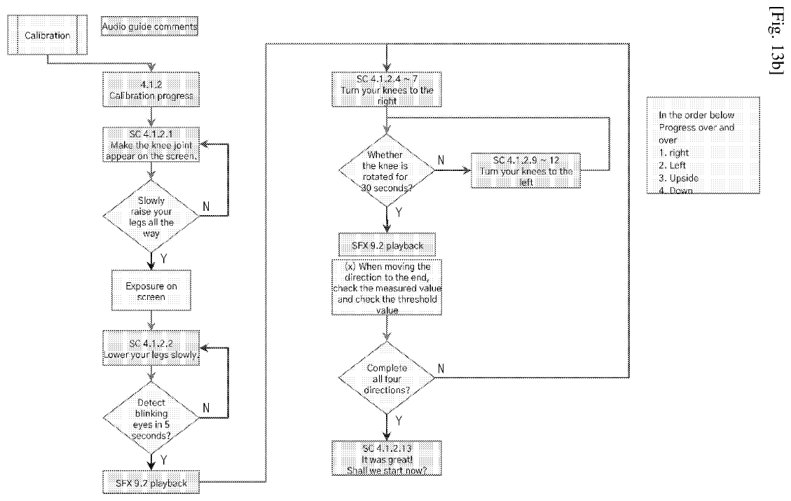

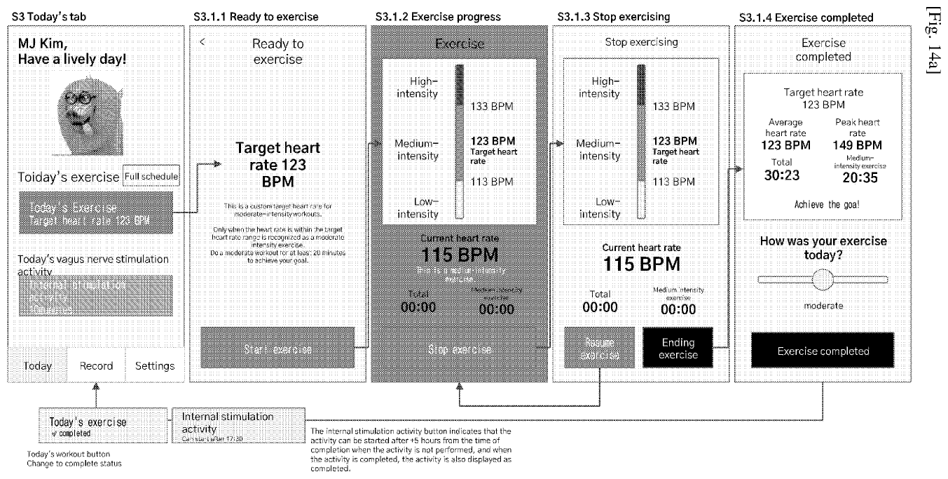

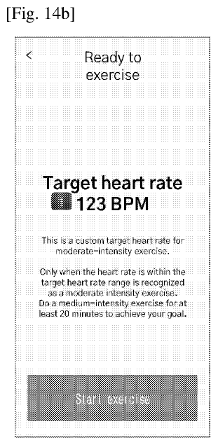

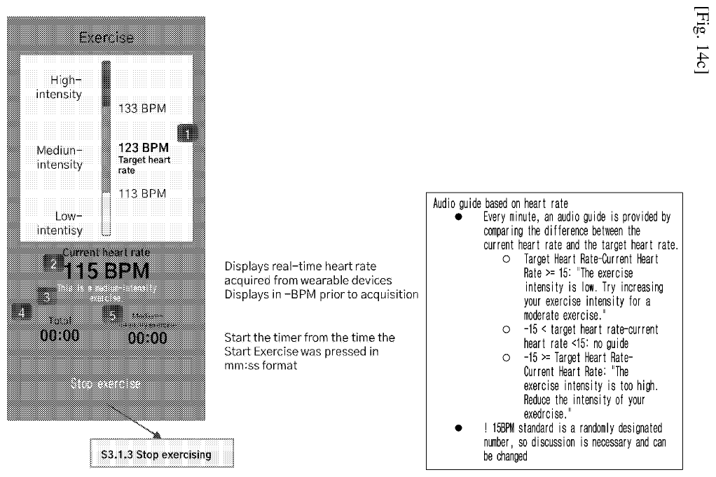

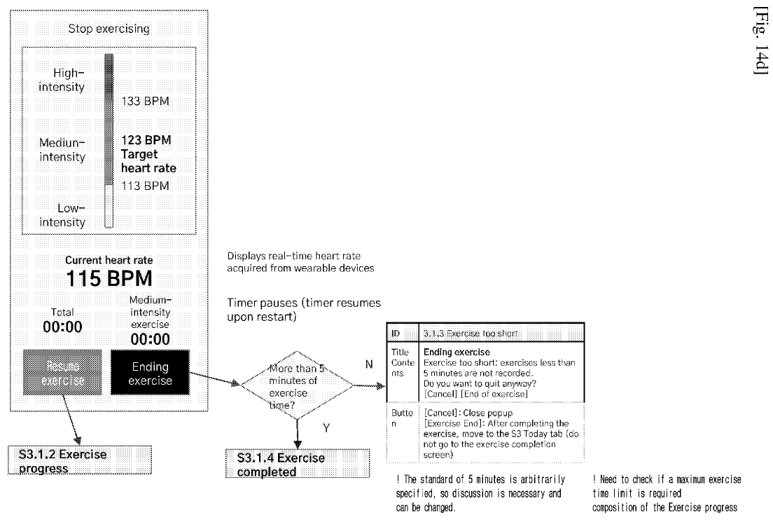

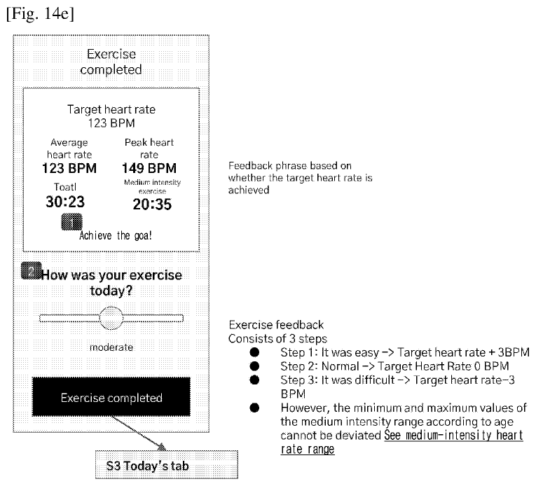

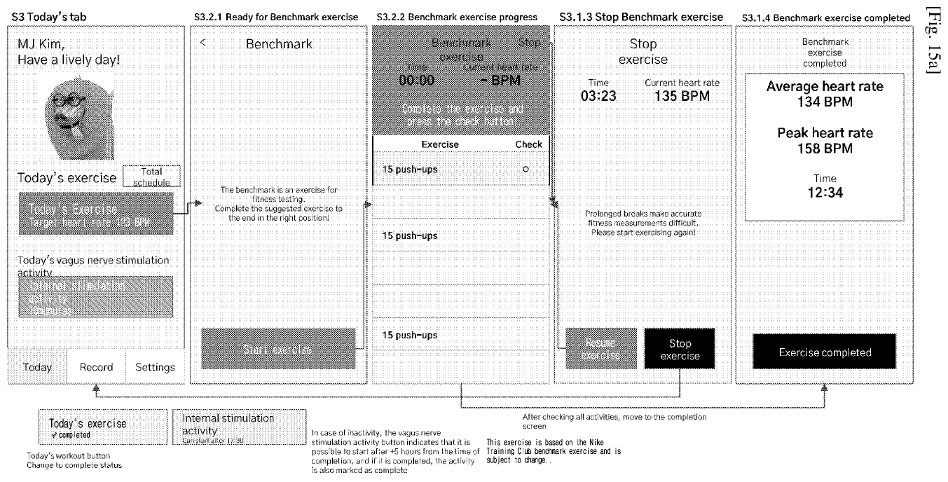

FIG. 13A depicts a calibration screen of a calibration module of a digital application of the present disclosure, wherein the calibration screen indicates a position that the subject's joint (e.g., a knee) should be positioned within a camera's field of view. As shown, (1) is a ready button for moving to calibration progress (4.1.2), (2) displays the screen exposure seen through the front camera, (3) shows a character of a posture, after the application recognizes the posture, the joint position is recognized. And, (4) shows information to the subject about the posture. FIG. 13B depicts a flow chart of the application execution flow for a calibration module of the present disclosure. For example, calibration may perform daily prior to the session start for angular measurement accuracy for about 60 seconds according to the result of instruction execution.

FIG. 14A depicts a medium-intensity exercise flow for a joint exercise module of the present disclosure. FIG. 14B depicts a screen of ready-to-exercise. As shown, (1) is a current target heart rate. The screen may further comprise information, for example, “Expose your target heart rate and get you ready for exercise.” FIG. 14C depicts a screen of exercise progress. As shown, (1) is a target heart rate display, at which +10 BPM and −10 BPM based on the subject's target heart rate are displayed as medium intensity exercise (caution, if the target heart rate is MAX, it can be a high-intensity exercise at +10 BPM), the whole image is used as a fixed, only target heart rate and the heart rate numbers displayed above and below are changed. The 10 BPM standard is a randomly designated number, so can be changed. (2) is a current heart rate, displaying the heart rate acquired through the wearable device. (3) displays high/medium/low intensity exercise by comparing the current heart rate and target heart rate. In embodiments, when the target heart rate is higher than +10 BPM, the exercise is a high intensity exercise. In embodiments, when the target heart rate is between −10 BPM and +10 BPM, the exercise is a medium intensity exercise. In embodiments, when the target heart rate is lower than −10 BPM, the exercise is a low intensity exercise. The text may not be displayed when heart rate measurement fails. (4) shows exercise full time timer. (5) shows a timer that accumulates only when the heart rate is in the medium intensity range. FIG. 14D depicts a screen of stop exercising. The screen may be displayed when exercise is paused. By design, it differentiates the progression of the exercise so that the subject can see at a glance where it is stopped. FIG. 14E depicts a screen of exercise completed. The screen shows a summary of the subject's exercise log when the workout is completed. And, the subject can also do exercise feedback. As shown, (1) is a goal achievement criteria. In embodiments, the goal is achieved when the heart rate is within the range of medium-intensity exercise (+−10 BPM based on the target heart rate) for 20 minutes or more. In embodiments, when reaching the goal, the screen further comprises the feedback phrase “Achieve the goal!” In embodiments, if the goal is not achieved, the phrase “the goal was not achieved” is displayed. Since the above criteria are randomly assigned numbers, so they can be changed. (2) shows Exercise intensity feedback. In embodiments, the application can ask questions about the subject's exercise intensity to calculate the subject's target heart rate. In embodiments, it is divided into 3 levels, and the default is normal. In embodiments, target heart rate calculations based on feedback follow the figures.

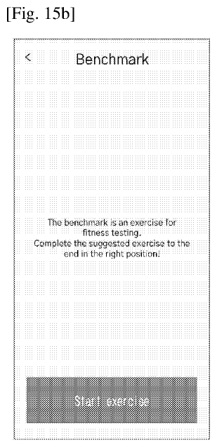

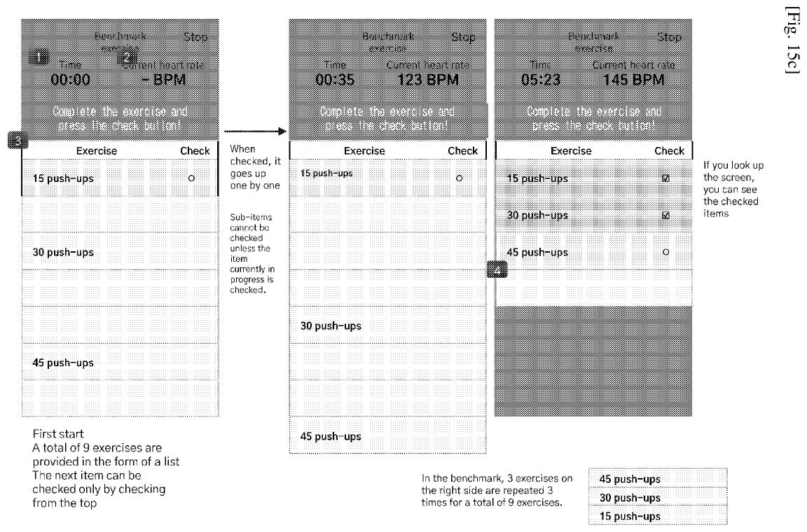

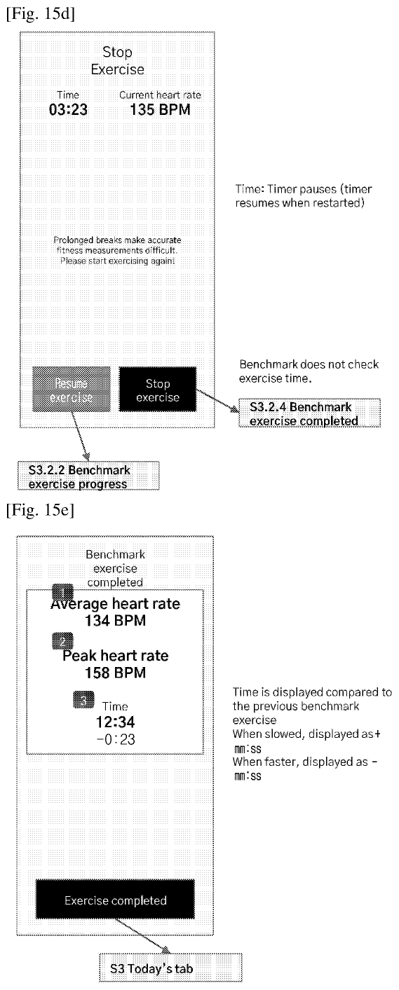

FIG. 15A depicts a benchmark exercise flow for a benchmark exercise module of the present disclosure. FIG. 15B depicts a screen of ready-for-benchmark exercise. As shown, benchmarking information may be displayed. FIG. 15C depicts a screen of benchmark exercise progress. Unlike medium-intensity exercise, the screen may present a designated exercise and the subject can check it when the exercise is completed. (1) shows a running time. In embodiments, it displays the progression time from the moment the subject started the benchmark. In embodiments, it is mm:ss format. In embodiments, time stops when paused. (2) shows a current heart rate. (3) An audio guide can be provided according to exercise progress. In embodiments, when subject check the last push-up, the audio “Thank you!” is sounded. In embodiments, the screen changes after the audio guide ends. (4) When the last exercise is checked, the screen changes to S3.2.4 Benchmark exercise completed. FIG. 15D depicts a screen of stop benchmark exercising. The screen may be displayed when exercise is paused. The screen when the exercise is paused may be designed to differ from the exercise progression so that the subject can grasp the paused state at a glance. FIG. 15E depicts a screen of benchmark exercise completed. The screen shows a summary of the subject's exercise log when the workout is completed. And, the subject can also do exercise feedback. As shown, (1) is an average heart rate. (2) is a peak heart rate. In embodiments, the highest heart rate recorded during exercise. (3) shows a time. In embodiments, the time is displayed compared to the previous benchmark, for example, when slowed, it is displayed as +00:12, when faster, it is displayed as −00:12. However, time comparison may not be performed at the initial benchmark. In embodiments, target heart rate may be changed according to recording range. For example, when the time is faster over 10 seconds, the target heart rate may be changed by +3 BPM. When the time is faster under 10 seconds and slower under 10 seconds, the target heart rate may be not changed. When the time is slower than 10 seconds, the target heart rate may be changed by −3 BPM. However, since there is no record to compare at the initial benchmark, the target heart rate may be not changed. 10 seconds and 3 BPM standards are randomly assigned numbers, so they can be changed.

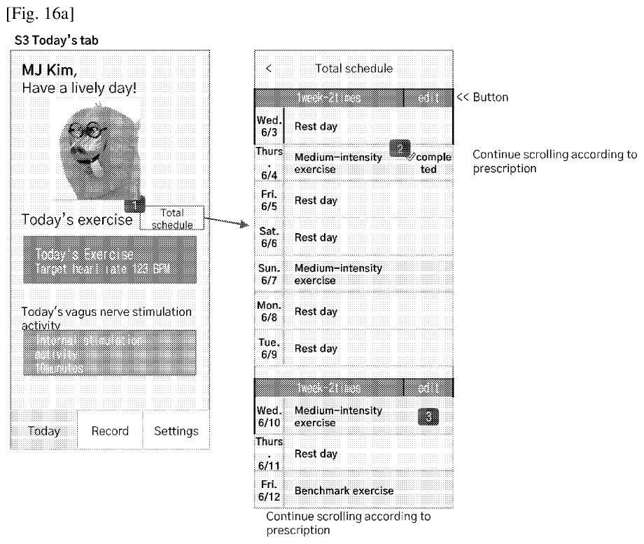

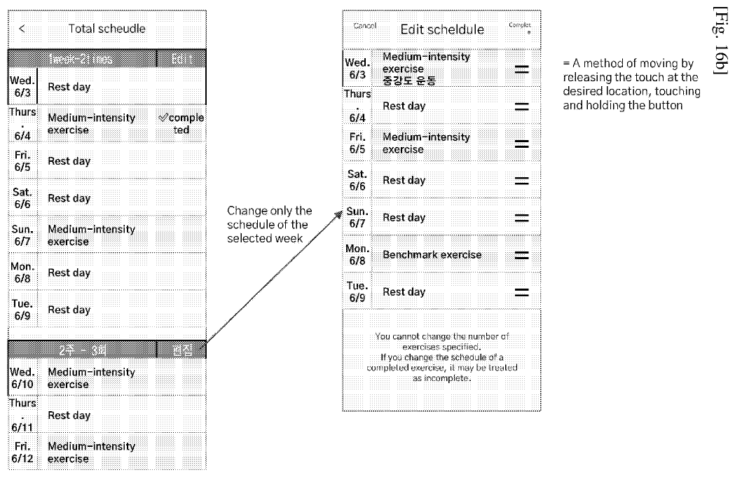

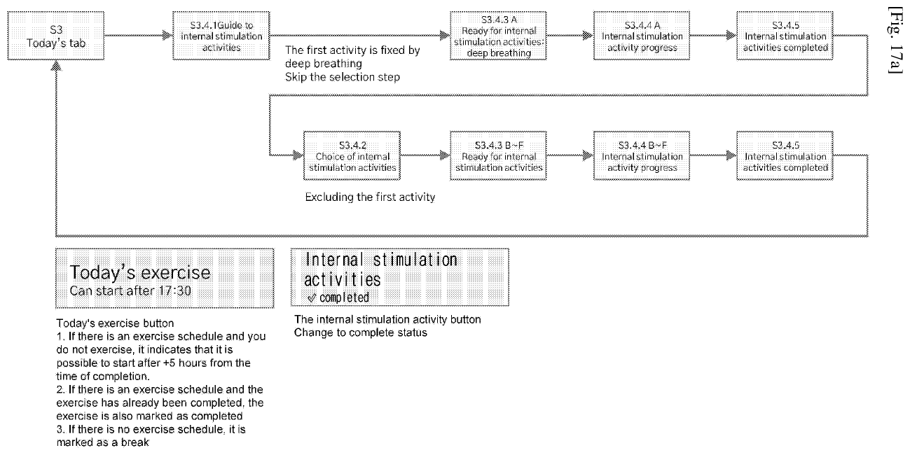

FIG. 16A depicts a schedule screen of a digital application of the present disclosure. The screen shows only the schedule of the current prescription. (1) is a total schedule button. Referring to (2), criteria for checking exercise completion will be described. In embodiments, exercise schedule and actual exercise are separated. In embodiments, it checks if there is an exercise record for the day, and if there is a record, it is marked as completed, if not, it is not displayed. It doesn't matter if the subject has reached the subject's goal. For example, if the subject moves an exercise marked as 6/4 complete, to 6/3 without exercise, the 6/3 exercise is not displayed as incomplete. At this time, if the subject moves the 6/3 exercise to the 6/4 exercised again, it is marked as complete. Referring to (3), criteria for checking benchmark exercise will be described. In embodiments, if the subject has a benchmark exercise schedule for that day, and the subject actually did the benchmark exercise, it will be marked as completed. In embodiments, if the subject does not perform a benchmark exercise on a day with a benchmark exercise schedule and only proceed with a normal medium-intensity exercise, there is an indication as incomplete. FIG. 16B depicts a screen of edit schedule. Among the weekly schedules, the subject can change the schedule.

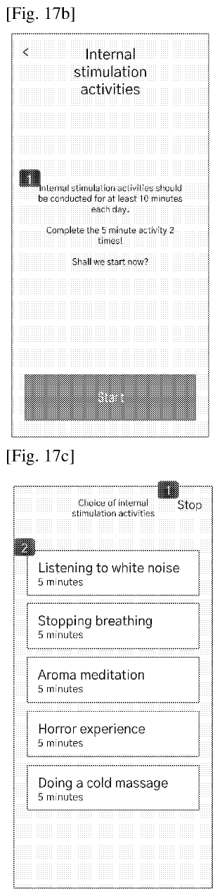

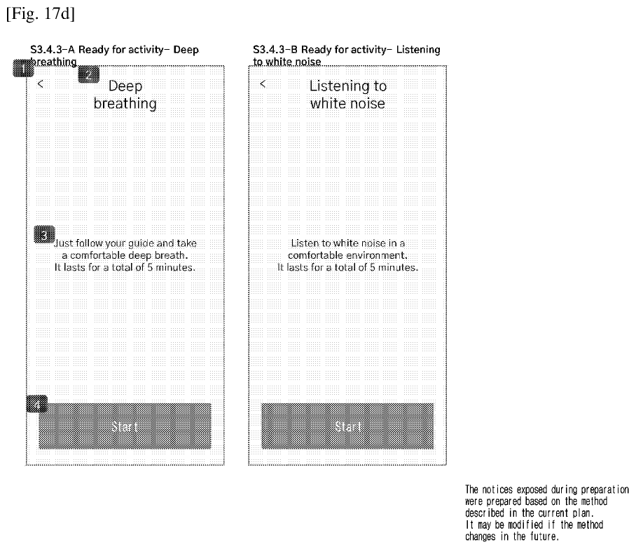

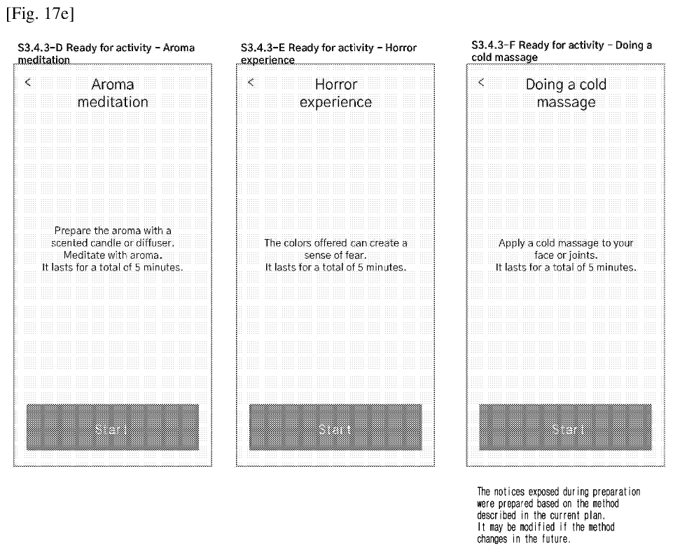

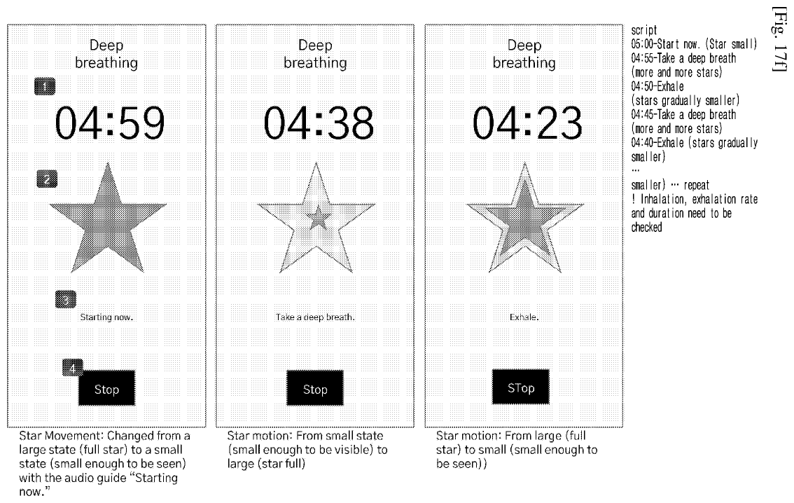

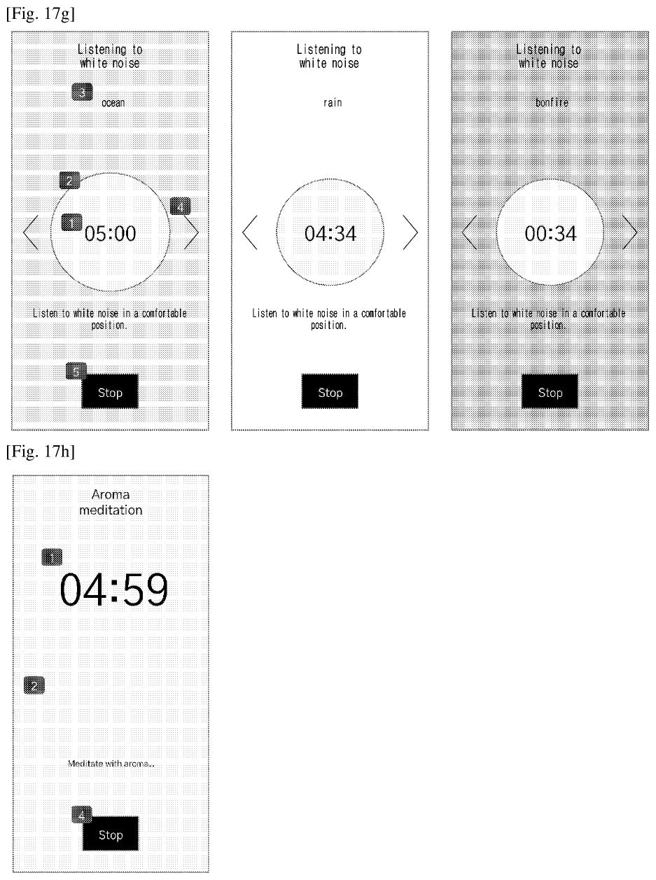

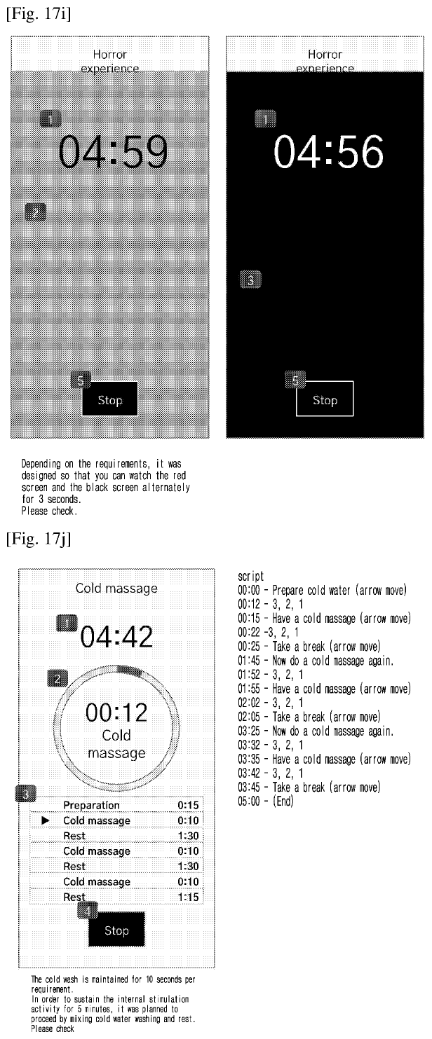

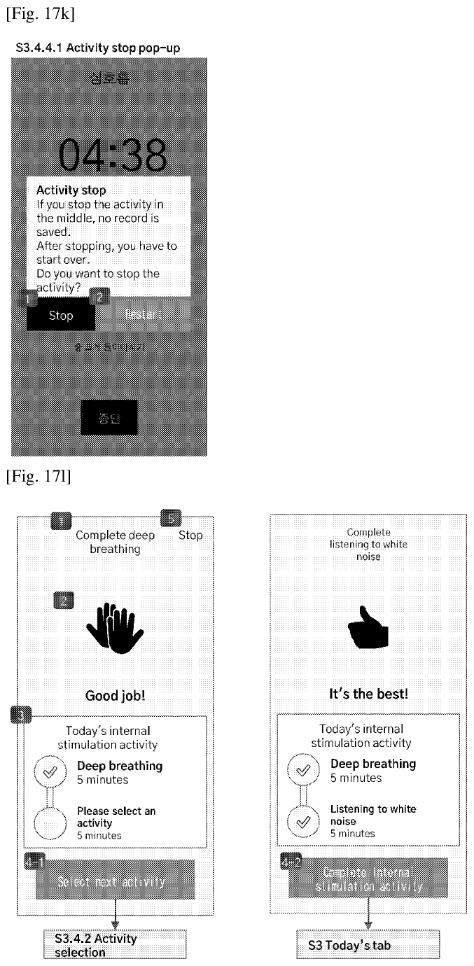

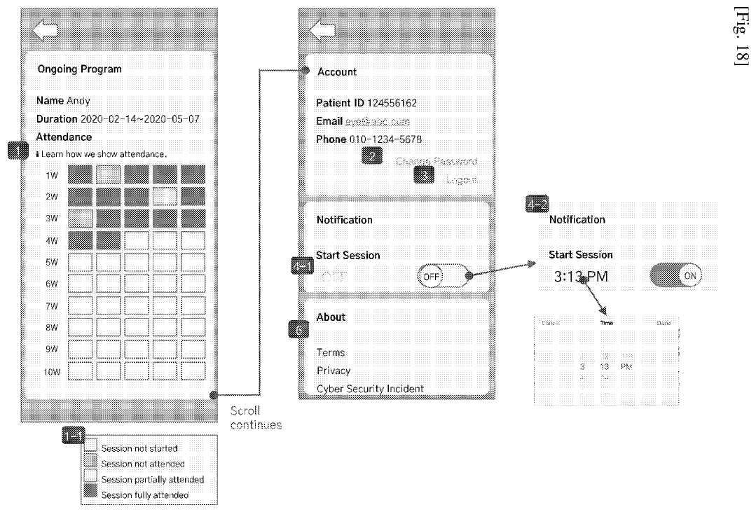

FIG. 17A depicts a flow chart of the application execution flow for a vagal nerve stimulation module of the present disclosure.

FIG. 17B depicts a screen guiding to internal stimulation activities. In embodiments, a total of 10 minutes is required (two times in a row), so the restrictions will be announced in advance.

FIG. 17C depicts a screen for choice of internal stimulation activities. The screen lists the selectable activities to allow the patient to choose. (1) is a stop button, when it tapped, S3.4.4.1 Activity stop pop-up may be exposed. (2) shows activity list. Activities selected at one time may be excluded.

FIG. 17D depicts a screen for ready to internal stimulation activities (deep breathing/listening to white noise/stopping breathing). In embodiments, different activities may have different titles and instructions. The overall structure may be the same for all activities. (1) is a previous button. When it tapped, the application moves to S3.4.2 activity selection. (2) shows a title, displays the name of the activity. (3) shows guidance phrase, in embodiments, different activities may display content differently. (4) is a start button, common to all activities, and when it is tapped, the application changes to S3.4.4 activity progress.

FIG. 17E depicts a screen for ready to internal stimulation activities (Aroma meditation/Horror experience/Doing a cold massage).

FIG. 17F depicts a screen of internal stimulation activity progress (deep breathing progress). As shown, (1) is a timer, for example, 5-minute timer. The timer may be started immediately upon entering the screen. Automatic screen switching may be provided at the end of the 5-minute timer. (2) shows a star. It doesn't have to go ahead with a star shape. The size of the star may be varied according to the progress of a deep breath. During breathing in, a small star may be changed to a big star. During exhale, a big star may be changed to a little star. (3) shows a guidance phrase (An audio guide may be provided too). In embodiments, guidance phrases and audio guides are provided as the subject progress in deep breathing. (4) is a stop button. When it is tapped, S3.4.4.1 stop pop-up may be exposed. When stopped, everything on the screen may be paused. Elements that were paused when resumed from the stop pop-up restart.

FIG. 17G depicts a screen of internal stimulation activity progress (white noise listening progress). As shown, (1) is a timer, for example, 5-minute timer. The timer may be started immediately upon entering the screen. Automatic screen switching may be provided at the end of the 5-minute timer. (2) shows a circle. It is not necessary to proceed in a circle shape. As the white noise listening progresses, it may increase or decrease at a constant tempo. (3) shows a theme of the currently playing sound. (4) may be a button to change the sound theme. In embodiments, it provides a total of 4 sounds. Swiping the screen or touching the arrows may change the themes in order. The theme change may be independent of the timer. Themes and resources may be a wave sound, rain sound, bonfire sound, forest sound, etc. Music may be less than 5 minutes needs to be played repeatedly. When changing the theme, it is okay to change the wallpaper as well. (5) is a stop button. When it is tapped, S3.4.4.1 stop pop-up may be exposed. When stopped, everything on the screen may be paused. When resumed from the pause, timer and audio that were paused pop-up restart.

FIG. 17H depicts a screen of internal stimulation activity progress (Aroma meditation progress). As shown, (1) is a timer, for example, 5-minute timer. The timer may be started immediately upon entering the screen. Automatic screen switching may be provided at the end of the 5-minute timer. (2) may provide a background music, for example, resource-Vagus Nerve_Aroma Meditation_bgm.mp3. (4) is a stop button. When it is tapped, S3.4.4.1 stop pop-up may be exposed. When stopped, everything on the screen may be paused. Elements that were paused when resumed from the stop pop-up restart.

FIG. 17I depicts a screen of internal stimulation activity progress (Horror experience progress). As shown, (1) is a timer, for example, 5-minute timer. The timer may be started immediately upon entering the screen. Automatic screen switching may be provided at the end of the 5-minute timer. (2) shows a red background, it may switch to black background after 3-second exposure. (3) shows a black background, it may switch to red background after 3-second exposure. It may repeat every 3 seconds. The application may provide a background music, for example, resource-Vagus Nerve_Aroma Meditation_bgm.mp3. It may repeat playback for 5-minutes. (5) is a stop button. When it is tapped, S3.4.4.1 stop pop-up may be exposed. When stopped, everything on the screen may be paused. Elements that were paused when resumed from the stop pop-up restart.

FIG. 17J depicts a screen of internal stimulation activity progress (Face immersion in cold water). As shown, (1) is a timer, for example, 5-minute timer. The timer may be started immediately upon entering the screen. Automatic screen switching may be provided at the end of the 5-minute timer. (2) shows a progress and step timer. In embodiments, preparation, cold massage, and relaxation may be performed in a total of 7 steps. Apart from the full timer, it may provide a timer for each step. (3) shows a display of all steps and steps currently in progress. The contents and time may displayed for a total of 7 steps, and the steps currently in progress may be marked with [symbol] . (4) is a stop button. When it is tapped, S3.4.4.1 stop pop-up may be exposed. When stopped, everything on the screen may be paused. Elements that were paused when resumed from the stop pop-up restart.

FIG. 17L depicts a screen of completion internal stimulation activity (Completion of deep breathing). It notifies the completion of the activity and provides the progress of the current vagus nerve stimulation activity. As shown, (1) displays the name of the completed activity. (2) displays images and phrases to congratulate the completion of the activity, for example, completion 1: Good job!, completion 2: It's the best!, etc. (3) shows the summary of today's internal stimulation activities. Separately displaying activities that have been performed and activities that have not may be provided. Activities that have been conducted may be displayed with the activity name and time. Activities not conducted may be exposed as “Please select an activity”, if the time is fixed at 5 minutes, “5 minutes” guide may be provided. (4-1) is a button, for when there is an activity to proceed (Only 1 out of 2 activities is proceeded). If next activity is selected, the screen switch to S3.4.2 select activity. (4-2) is a button, for When there is no activity to proceed (2 out of 2 activities are proceeded). When vagus nerve stimulation activity is completed, the screen switch to S3.today's tab. (5) is a stop button. When it is tapped, S3.4.4.1 stop pop-up may be exposed.

FIG. 18 depicts a screen of record mode (cold massage). As shown, (1) shows an attendance map information. (1-1) The tooltip may be provided upon click and be disappeared when tapped anywhere on the screen. (2) is a button for change the password, if it is tapped, the screen moves to ID 5.2.2. (2) is a button for logout, if it is tapped, the screen goes to the login screen. (4) is displayed for setting up the push notifications. In embodiments, push notification is possible once a day (except weekends). In embodiments, time settings may use an iOS's Time Picker UI. (4-1) When there is no push notification set up. (4-2) When there is a push notification set up, the time may be tapped to edit. In embodiments, a bright light authentication notification may be provided 3 times a day, for example, at 10 am, 2 μm, and 4 pm on weekdays. (6) shows an in-app web view (V1.5 item added). Items that require guidance, such as terms of service, privacy policy, and cybersecurity incident information, are processed through a web view to facilitating maintenance.

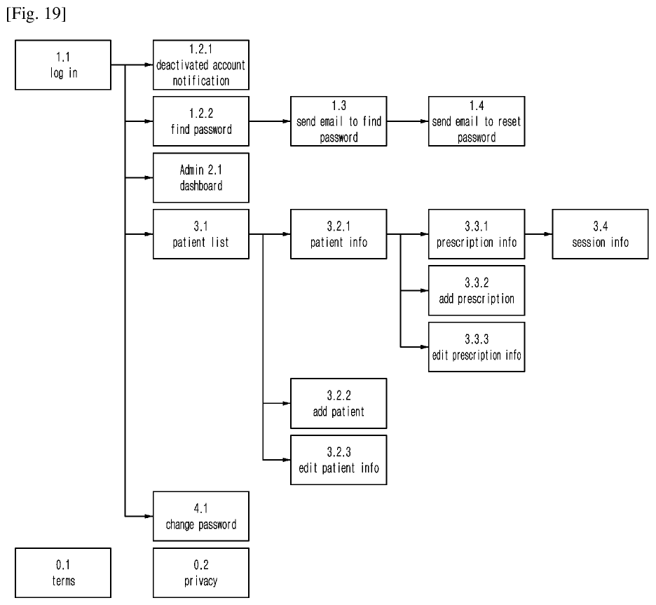

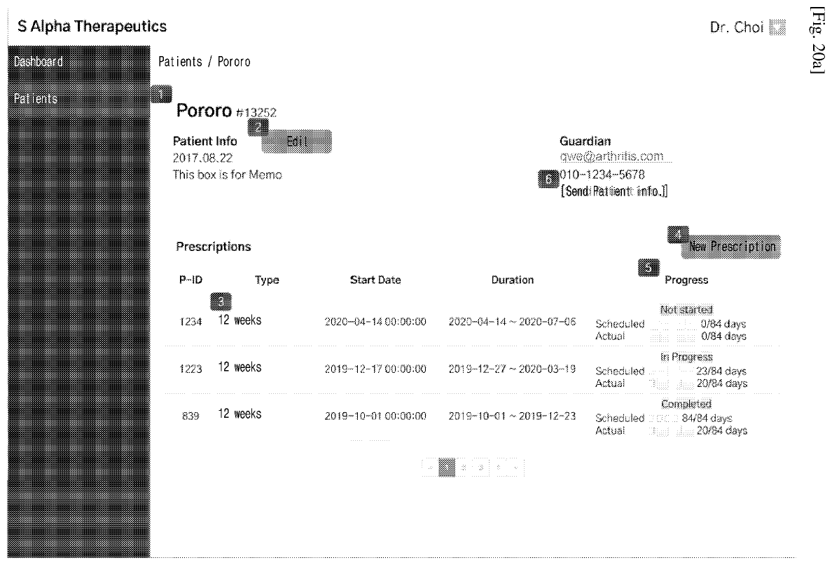

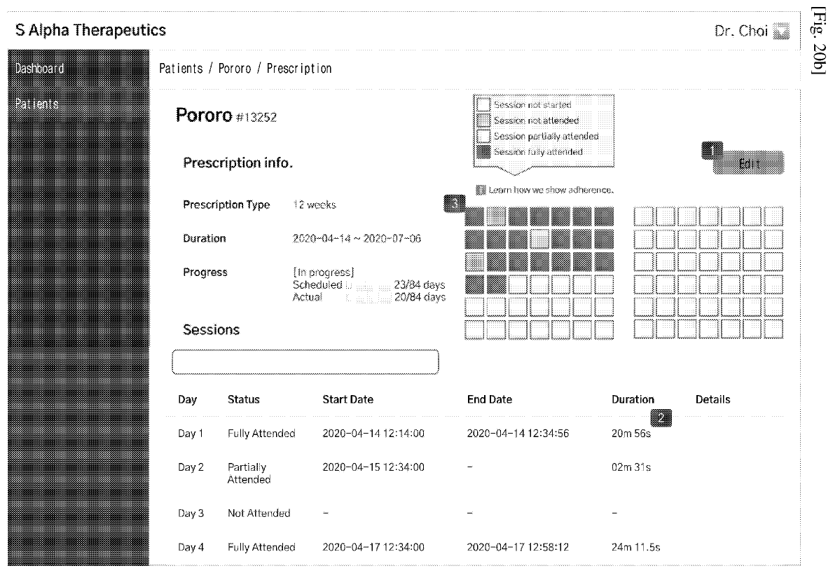

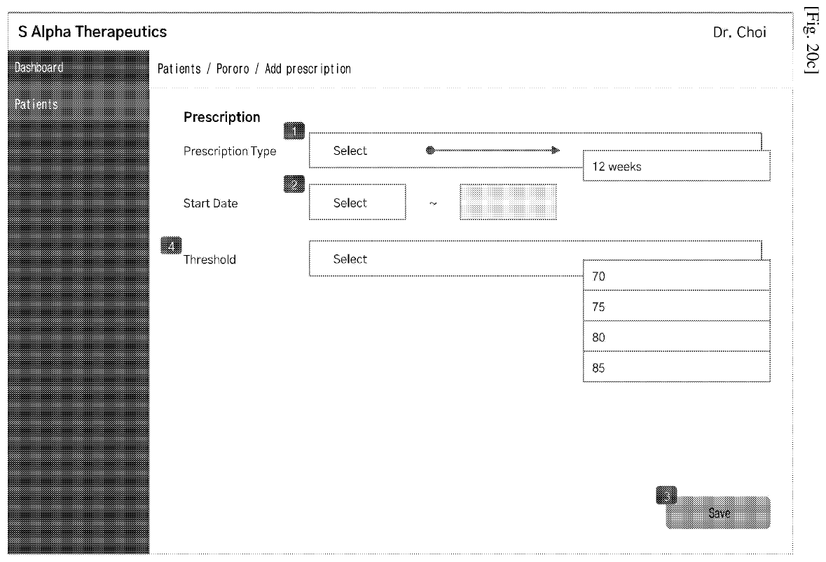

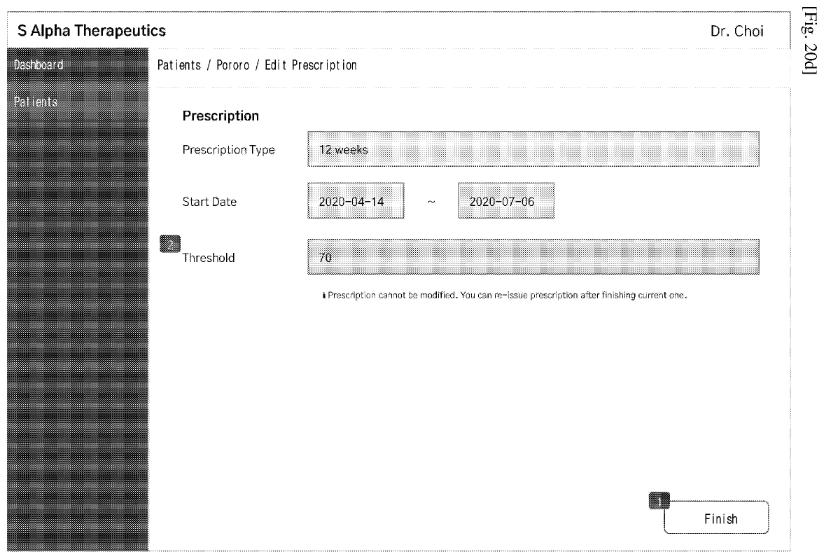

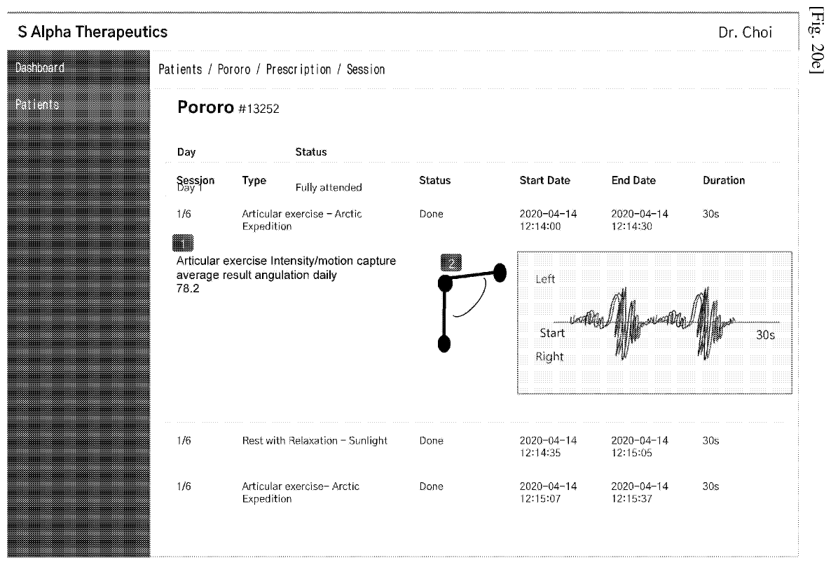

FIG. 19 is a flow chart illustrating an exemplary execution flow for a healthcare provider portal in a system of the present disclosure

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

FIGS. 20A-E depict (A) an exemplary patient tab in a healthcare provider portal, the patient tab displaying detailed information on a given patient, (B) an exemplary patient tab in a healthcare provider portal that displays detailed prescription information for a given patient, (C-D) an exemplary patient tab in a healthcare provider portal for editing prescription information for a given patient, and (E) a patient tab in a healthcare provider portal for viewing details (e.g., date, status, duration, results) of a given session for a given patient;

FIG. 20A depicts a patient tab in a healthcare provider portal, the patient tab displaying detailed information on a given patient. As shown, (1) detailed patient information, (2) a button for editing patient information, (3) prescription information, (4) a button for adding a new prescription, (5) displays a progress status for different each prescription, and (6) a button or link for sending an email to the patient.

FIG. 20B depicts a patient tab in a healthcare provider portal that displays detailed prescription information for a given patient. As shown, (1) is a button for editing prescription information, (2) displays the duration of the sessions attended by the patient or subject, and (3) shows an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended).

FIG. 20C depicts a patient tab in a healthcare provider portal for editing prescription information for a given patient (Add Patient Treatment). (1) Select treatment type. It may be changed after checking the actual data. (2) Select treatment period. The end date may not be arbitrarily modified and is fixed according to the treatment period and start date of the prescription. (3) Add treatment. When the button is clicked, the following items are checked in order. If the condition of each item is not satisfied, the item is displayed as shown in 2. and 3. Error toast pop-up is displayed. Check for ongoing treatment: “Unfinished prescription for this patient exists.” If the scheduled treatment and the selected treatment period overlap: “Treatment cannot proceed at the same time. Please check the start date.” No treatment type selected: “Select prescription type.” No treatment period selected: “Select start date.” (4) Threshold. It allows user to select a reference point for eye movement when prescribing. The higher the eye movement reference point may be, the more eye movement within the app is required to be recognized as movement. Threshold number is subject to change. Add treatment when all are met. Email/SMS sending: <Email-P2><SMS-P2>. Show success toast popup after moving to patient details “Added prescription”

FIG. 20D depicts a patient tab in a healthcare provider portal for editing prescription information for a given patient (Modify patient treatment information). This is the screen where the doctor can manage the patient's treatment. Modification may be not possible, only forced termination may be possible. (1) Forced termination of treatment. Show confirmation popup on click (for example, Finish this prescription? This cannot be undone. [Cancel] or [Finish]). When [Finish] is clicked on the pop-up, the treatment is forcibly terminated, moving to the patient details, and the success toast pop-up is exposed. “The treatment has ended.” end email/SMS after forced termination: <Email-P3><SMS-P3>. (2) Threshold added.

FIG. 20E depicts a patient tab in a healthcare provider portal for viewing details (e.g., date, status, duration, results) of a given session for a given patient. This is a screen where user can check the details of each session. (1) The formula for calculating the average joint motion value. When the measured joint position (x, y) is mapped on the coordinates of (−100,−100)˜(100, 100), let d be the distance from (0, 0). (Absolute value). When the joint position is measured every 100 ms during the joint exercise time excluding the rest time, if this value is d1˜dN, then sum(d1 . . . dN)/N is the average intensity. Since this value can have a value of 0˜144.xyz. The final average intensity divided by 145 and multiplied by 100 becomes the exercise intensity. (2) Joint motion graph. Classify top/bottom/left/right by color, for example, Up and Down-Red/Left and Right-Blue. In the top, a + means “top”, and a − means “bottom”. On the right, + means “right”, and − means “left”. Calculate exercise intensity as 0˜100 by calculation formula. The amplitude is large=> The joint has moved a lot.

In further embodiments, a healthcare provider portal may provide a dashboard. The dashboard may display the number of all patients associated with the present doctor's account. The dashboard may display a graph that may be used to show the number of patients who have opened the digital application for patient per day in the most recent 90 days. The number of patients in progress may also be viewed. A graph may be used to show the number of patients who have completed the daily sessions per day in the most recent 90 days. In embodiments, a healthcare provider portal may provide a patient tab, the patient tab may display a list of patients. The patient tab may display a Patient ID (the unique identification number temporarily given to each patient when adding them on the list), a Patient Name, a Search bar for searching by ID, Name, Email, Memo, etc., and an Add New Patient button for adding new patients. In further embodiments, a healthcare provider portal may provide a patient tab for adding a new patient. The patient tab may provide a button for adding a new patient, and an error message may be displayed when required patient information has not been provided. In further embodiments, a healthcare provider portal may provide a patient tab for editing information of an existing patient. The patient tab may provide a button or link for resetting a password, a button for deleting a given patient, and a button for saving changes.

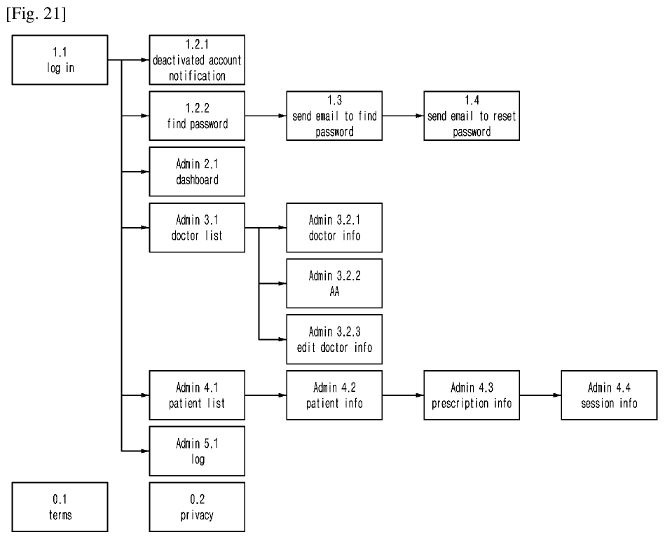

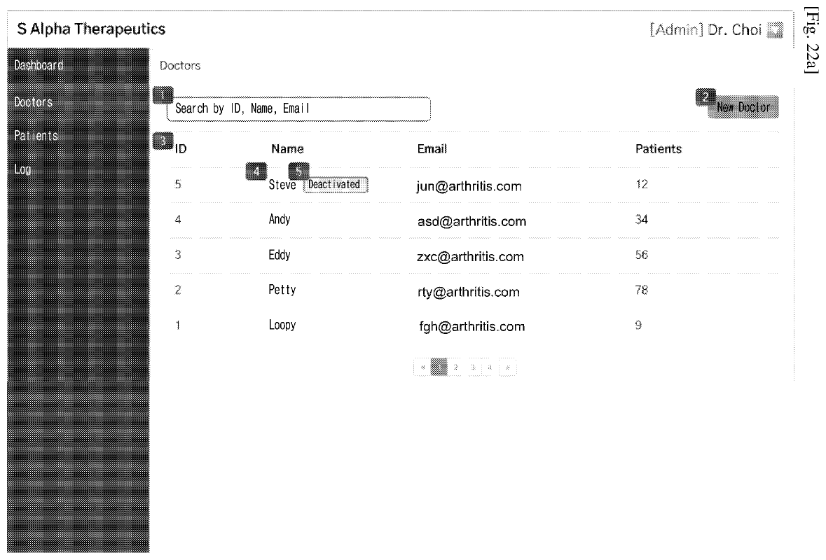

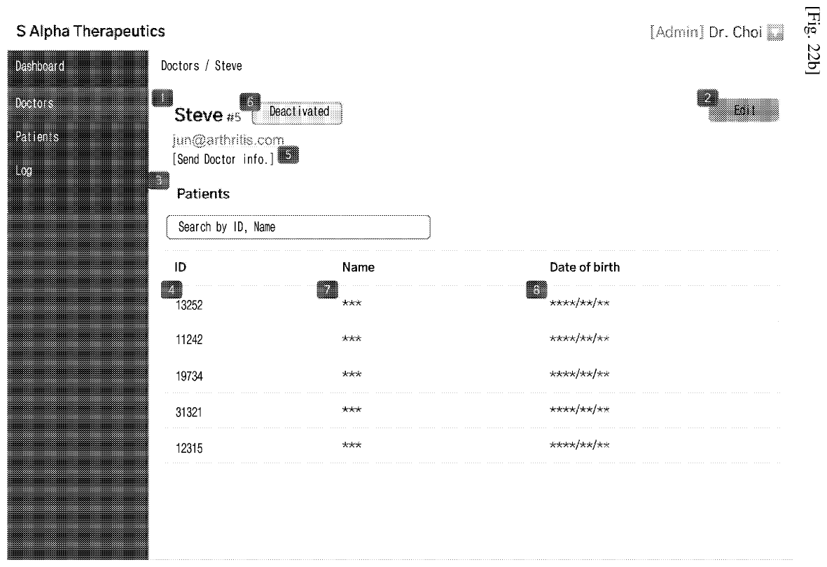

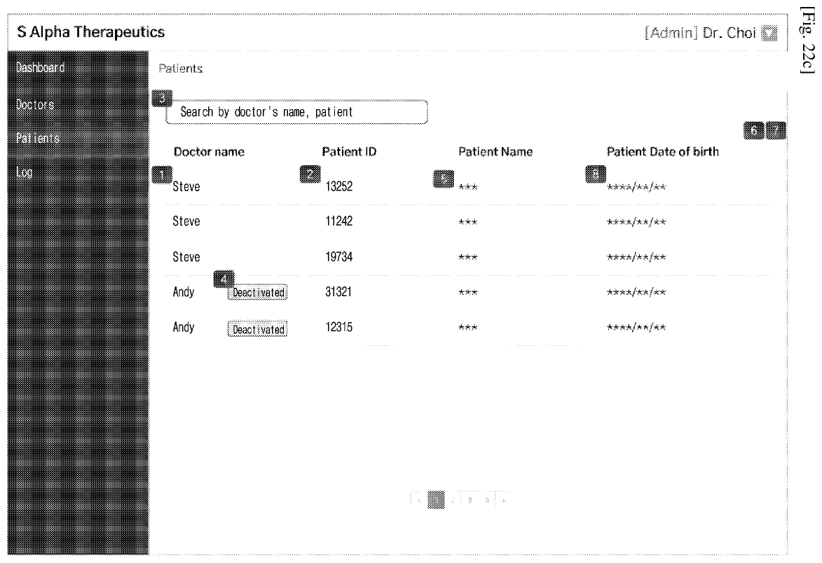

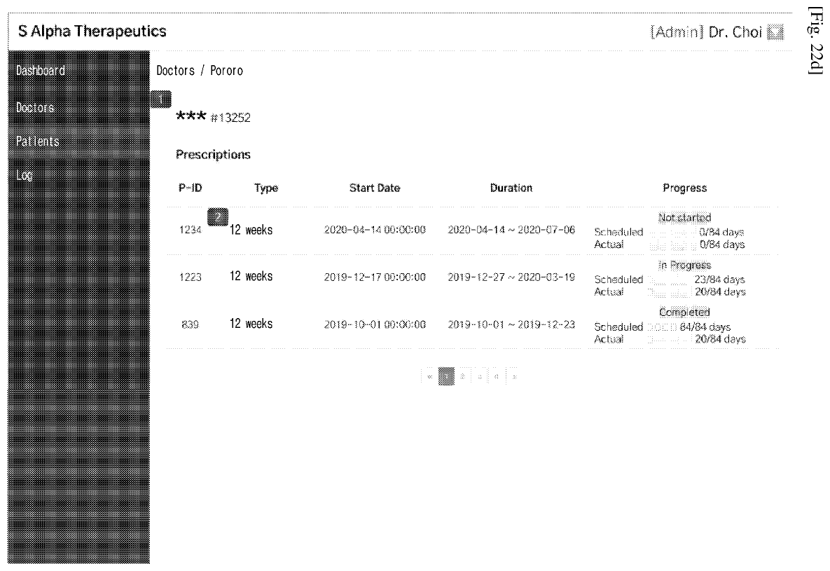

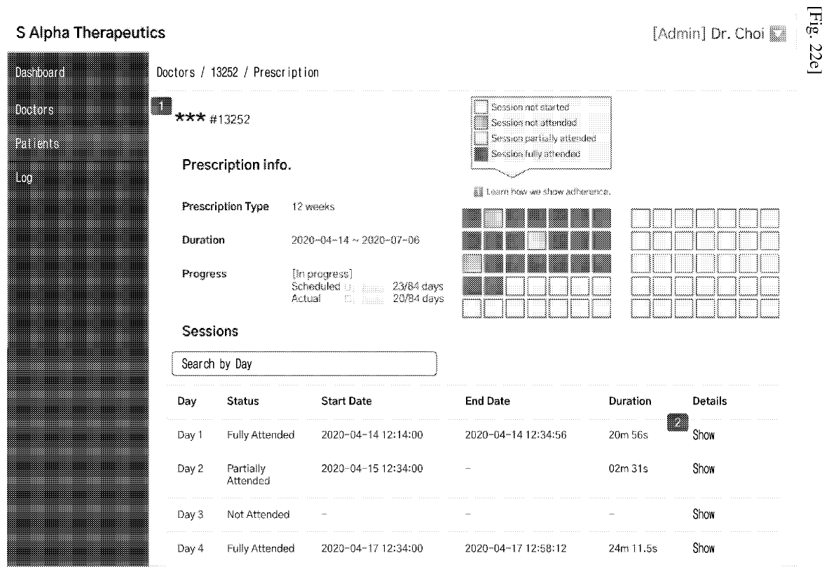

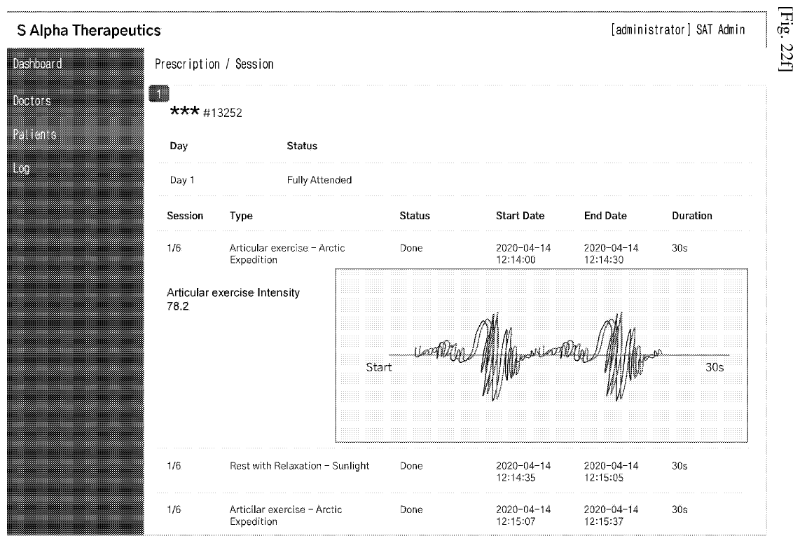

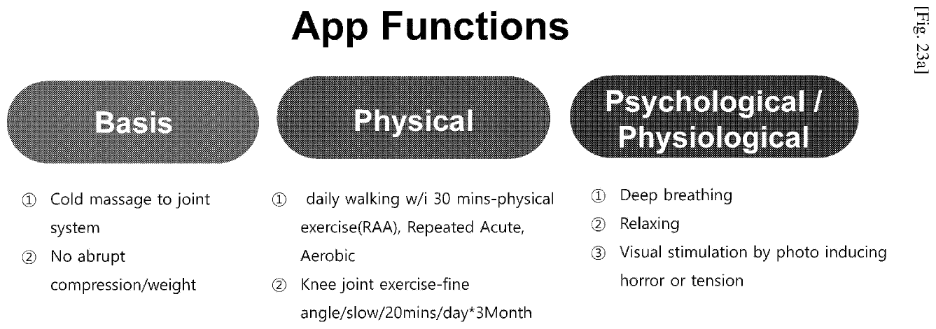

FIG. 21 is a flow chart illustrating an exemplary execution flow for an administrative portal in a system of the present disclosure.

In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

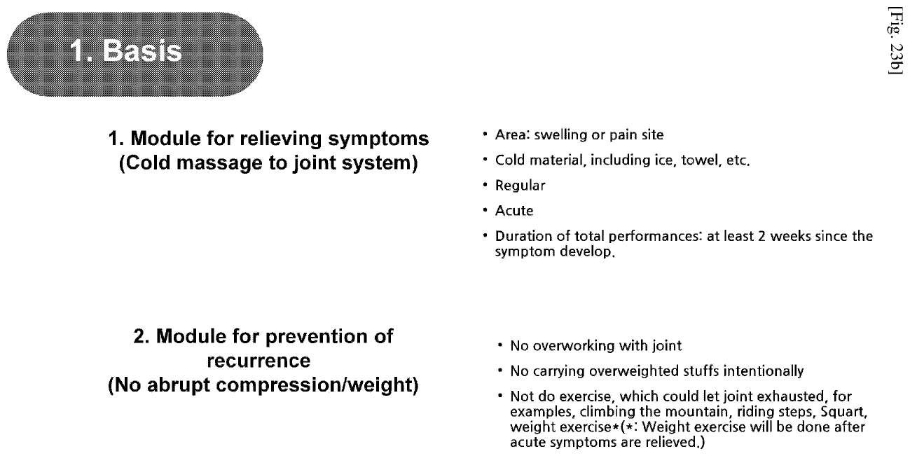

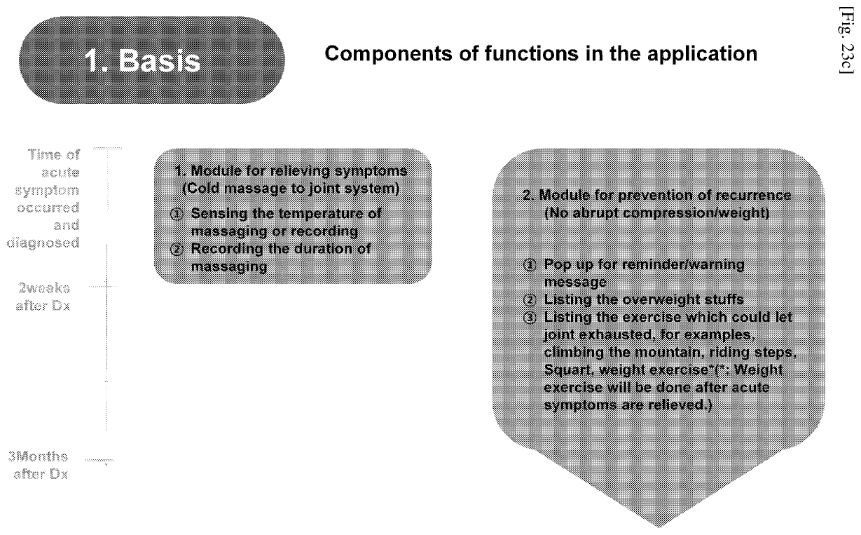

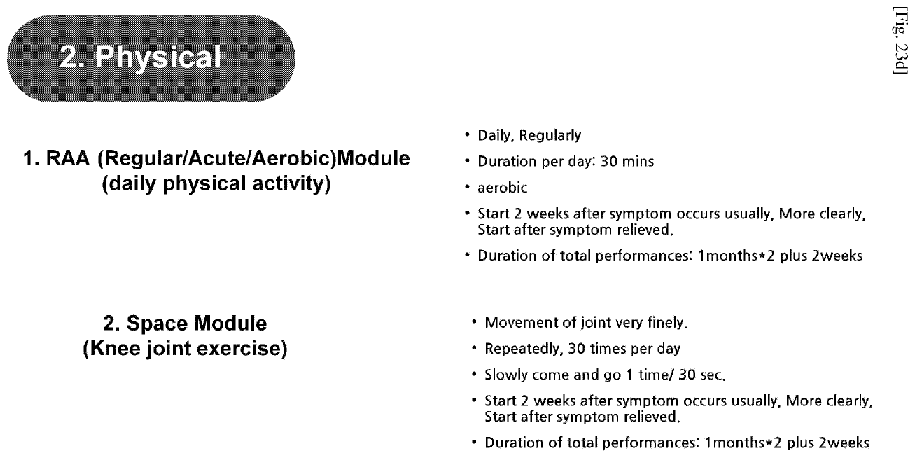

FIG. 22A depicts a doctor tab in an administrative portal, the doctor tab displaying a list of doctors. As shown, (1) is a search bar for searching for various doctors by name, email, etc., (2) shows a button for adding a new doctor, (3) is the doctor's ID, (4) is a button for viewing detailed doctor information, and (5) shows deactivated doctor accounts.

FIG. 22B depicts a doctor tab in an administrative portal, the doctor tab displaying a list of patients being cared for by a given doctor, with patient-identifying information redacted (*). As shown, (1) is the doctor's account information, (2) is a button for editing the doctor's account information, (3) is a list of patients being cared for by the doctor, (4) is a list of patient ID numbers, (5) a link or button for sending the doctor a registration email, (6) a notification that the doctor's account has been deactivated, which only appears for deactivated accounts, and (7 and 8) redacted or de-identified patient information.

FIG. 22C depicts a patient tab in an administrative portal that displays information for one or more patients, wherein sensitive information is redacted. FIG. 22D depicts a patient tab in an administrative portal that displays detailed patient or prescription information for a given patient. FIG. 22E depicts a patient tab in an administrative portal that displays detailed prescription information for a given patient. FIG. 22F depicts a patient tab in an administrative portal for viewing details (e.g., date, status, duration, results) of a given session for a given patient. This is a screen where the user can check the details of each session. The elements other than the de-identification process may be the same as in the Web 3.4 for doctors. De-identification treatment that all characters are treated to ‘*’ may be performed.

In further embodiments, an administrative portal may provide a dashboard. The dashboard may display the number of doctors. The dashboard may display a graph that may be used to show the number of doctors that have visited the digital application per day in the most recent 90 days, The number of all patients associated with the any doctor's account. A graph may be used to show the number of patients who have opened the digital application for patient per day in the most recent 90 days. The number of patients in progress may also be viewed. A graph may be used to show the number of patients who have completed the daily sessions per day in the most recent 90 days. In further embodiments, an administrative portal may provide a doctor tab for adding a new doctor. In further embodiments, an administrative portal may provide a doctor tab for editing information of an existing doctor, including activating or deactivating a doctor's account.

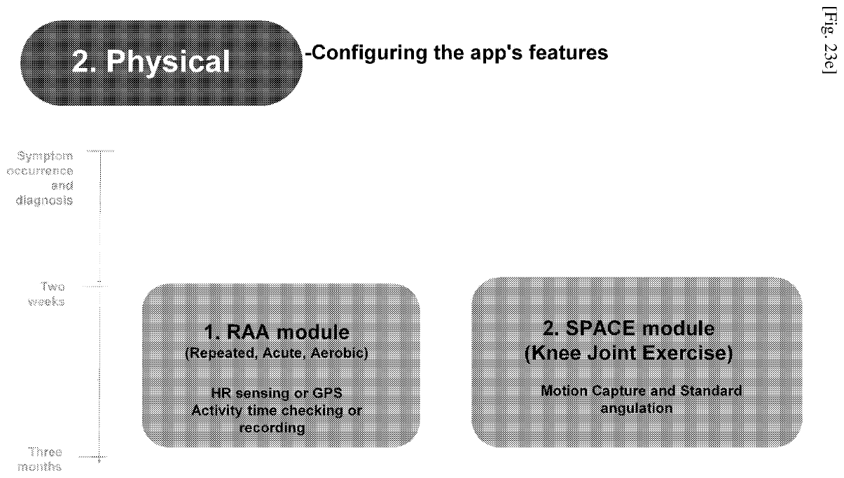

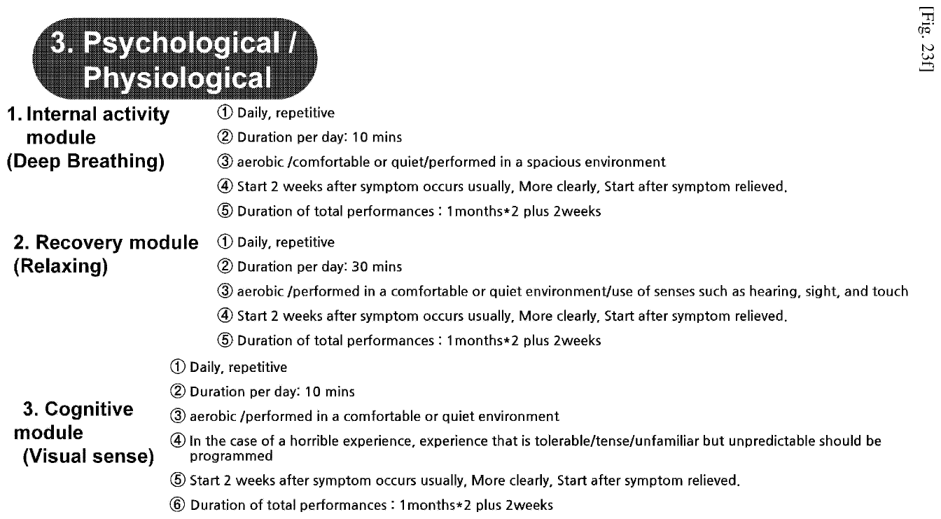

FIGS. 23A-G illustrates various application functions, including modules that may be performed by the subject, for example, to relieve pain symptoms, prevent recurrence of pain/disease, strengthen a joint, and/or induce relaxation; and

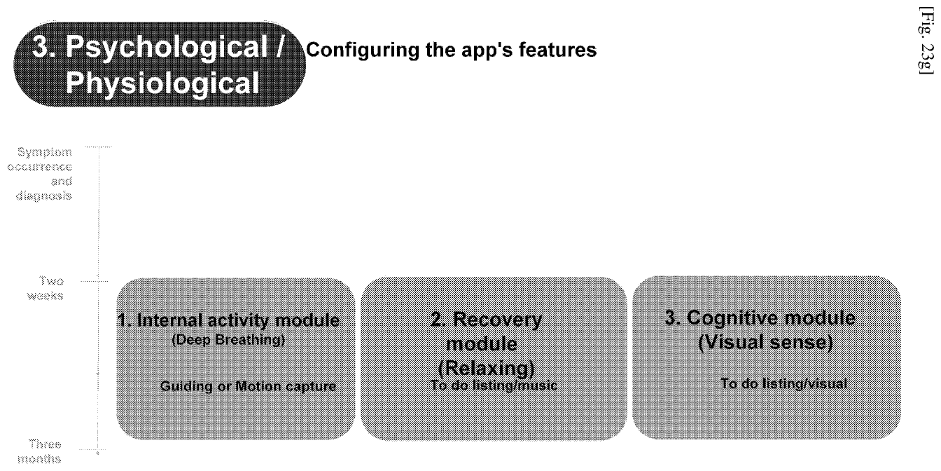

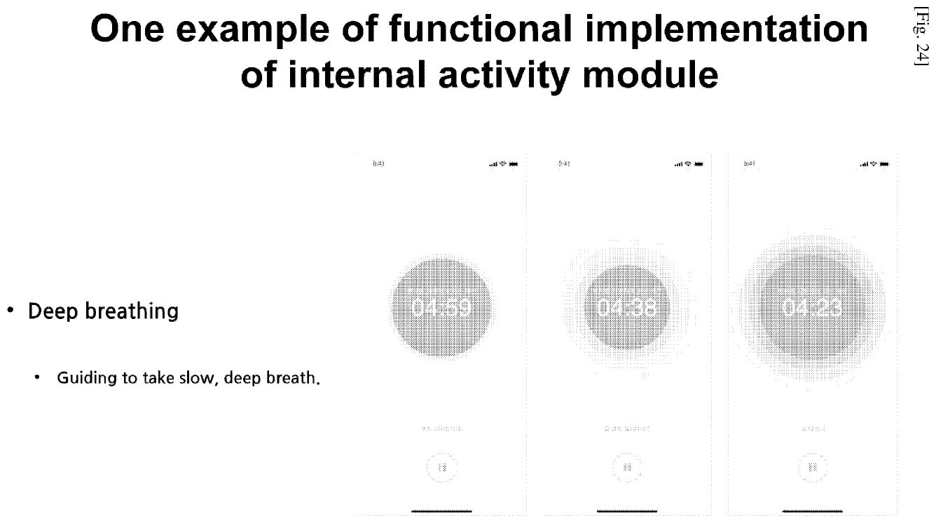

FIG. 24 illustrates an exemplary deep breathing exercise of the present disclosure.

While the disclosure has been shown and described with reference to certain exemplary embodiments thereof, it will be understood by those skilled in the art that various changes in form and details may be made therein without departing from the spirit and scope of the disclosure as defined by the appended claims.

### CERTAIN EMBODIMENTS

Embodiment 1. A method of preventing or ameliorating arthritis in a joint of a subject, the method comprising: providing, by an electronic device to the subject, one or more first modules selected from the group consisting of a vagal nerve stimulation module, a joint exercise module, and an adiponectin secretion module, each of the one or more first modules comprising one or more first instructions for the subject to follow; sensing, using a sensor of the electronic device, adherence by the subject to the first instructions of the one or more first modules, wherein the electronic device transmits adherence information, based on the adherence, to a server, and receives one or more second instructions from the server based on the adherence information, providing, by the electronic device to the subject, one or more second modules selected from the group consisting of an adiponectin secretion module, a vagal nerve stimulation module, and a joint exercise module, each of the one or more first modules comprising one or more second instructions.

Embodiment 2. The method of embodiment 1, wherein the arthritis is selected from the group consisting of osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

Embodiment 3. The method of embodiment 1 or 2, wherein the subject is an adult.

Embodiment 4. The method of any one of embodiments 1-3, wherein the one or more first modules comprise the vagal nerve stimulation module, and the vagal nerve stimulation module comprises one or more first instructions to reduce inflammation in the joint of the subject and/or increase the production of anti-inflammatory factors in the joint.

Embodiment 5. The method of any one of embodiments 1-4, wherein the one or more first modules comprise the vagal nerve stimulation module, and the vagal nerve stimulation module comprises at least one instructions selected from the group consisting of sense stimulation instructions for sight, sound, touch, taste, and smell.

Embodiment 6. The method of embodiment 5, wherein the vagal nerve stimulation module comprises one or more sense stimulation instructions for sight, and the one or more sense stimulation instructions for sight include one or more instructions to view one or more figures to stimulate autonomic nervous system.

Embodiment 7. The method of embodiment 6, wherein the electronic device receives and displays the figures.

Embodiment 8. The method of any one of embodiments 5-7, wherein the vagal nerve stimulation module comprises one or more sense stimulation instructions for sound, and the one or more sense stimulation instructions for sound include one or more instructions to hear one or more sounds to cause honor or relaxation.

Embodiment 9. The method of embodiment 8, wherein the electronic device receives and plays the sounds.

Embodiment 10. The method of any one of embodiments 5-9, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

Embodiment 11. The method of any one of embodiments 5-10, wherein the vagal nerve stimulation module comprises one or more sense stimulation instructions for touch, and the one or more sense stimulation instructions for touch include one or more instructions for abdominal breathing.

Embodiment 12. The method of any one of embodiments 5-11, wherein the vagal nerve stimulation module comprises one or more sense stimulation instructions for taste, and the one or more sense stimulation instructions for taste include one or more instructions to eat food to stimulate digestive glands in the subject.

Embodiment 13. The method of embodiment 12, wherein the electronic device receives and display information related to the food.

Embodiment 14. The method of any one of embodiments 5-13, wherein the vagal nerve stimulation module comprises one or more sense stimulation instructions for smell, and the one or more sense stimulation instructions for smell include one or more instructions to stimulate digestive glands and/or to relax.

Embodiment 15. The method of embodiment 14, wherein the electronic device is configured to release a scent for aroma therapy.

Embodiment 16. The method of any one of embodiments 1-15, wherein the one or more first modules do not comprise one or more first instructions for a cold massage.

Embodiment 17. The method of any one of embodiments 1-16, wherein the one or more first modules comprise the joint exercise module, and the joint exercise module comprises one or more first instructions to reduce inflammation in the joint of the subject.

Embodiment 18. The method of any one of embodiments 1-17, wherein the joint of the subject is selected from the group consisting of a shoulder joint, an elbow joint, a sacroiliac joint, a hip joint, a knee joint, a joint of the wrist or hand, a joint of the ankle or foot, a neck joint, and an intervertebral joint.

Embodiment 19. The method of embodiment 17 or 18, wherein the joint exercise comprises one or more of walking, running, stretching, and bending the joint at a predetermined angle and/or speed.

Embodiment 20. The method of any one of embodiments 1-19, wherein the one or more first modules comprise the adiponectin secretion module, and the adiponectin secretion module comprises one or more first instructions to increase a secretion of adiponectin from adipose tissue.

Embodiment 21. The method of embodiment 20, wherein the one or more first instructions to increase the secretion of adiponectin from adipose tissue are selected from the group consisting of walking, biking, aerobic dance, and swimming.

Embodiment 22. The method of any one of embodiments 1-21, wherein the server receives the one or more second instructions from an external reviewer.

Embodiment 23. The method of any one of embodiments 1-22, wherein the external reviewer comprises a health professional.

Embodiment 24. The method of any one of embodiments 1-23, wherein the external reviewer comprises an artificial intelligence (AI).

Embodiment 25. The method of any one of embodiments 1-24, wherein the sensor comprises one or more selected from the group consisting of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 26. The method of embodiment 25, wherein the sensor comprises a camera, and wherein the electronic device is configured to detect an angle at which the subject is bending the joint.

Embodiment 27. The method of embodiment 26, wherein the adherence information comprises one or both of the angle at which the subject is bending the joint, and a difference between the angle at which the subject is bending the joint and a predetermined angle at which the subject is instructed to bend the joint.

Embodiment 28. The method of embodiment 27, wherein, if the difference between the angle at which the subject is bending the joint and the predetermined angle at which the subject is instructed to bend the joint is greater than a predetermined threshold, the method further comprises providing to the subject one or more additional instructions to adjust the angle at which the subject is bending the joint until the difference is less than or equal to the predetermined threshold.

Embodiment 29. The method of any one of embodiments 17-28, wherein the joint exercise module induces normalization of the balance between (i) sympathetic innervation on the synovial membrane, (ii) sensory innervation on the synovial membrane, and (iii) Oncostatin M production.

Embodiment 30. The method of any one of embodiments 1-29, further comprising administering to the subject one or more drugs for preventing or ameliorating arthritis.

Embodiment 31. The method of embodiment 30, wherein the one or more drugs are administered to the subject in an amount and/or frequency that is less than an amount and/or frequency prescribed to an average arthritis patient.

Embodiment 32. The method of any one of embodiments 1-29, wherein the method is performed without administering one or more drugs for preventing or ameliorating arthritis to the subject.

Embodiment 33. A system for preventing or ameliorating arthritis in a joint of a subject, comprising: a digital apparatus configured to execute a digital application comprising one or more first modules for preventing or ameliorating arthritis in a joint of the subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment for preventing or ameliorating arthritis in a joint of the subject based on information received from the digital application; and an administrative portal configured to provide one or more options to an administrator of the system to perform one or more tasks to manage access to the system by the healthcare provider.

Embodiment 34. The system of embodiment 33, wherein the arthritis is selected from the group consisting of osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

Embodiment 35. The system of embodiment 33 or 34, wherein the subject is an adult.

Embodiment 36. The system of any one of embodiments 33-35, wherein the one or more first modules comprise the vagal nerve stimulation module, and the vagal nerve stimulation module comprises one or more first instructions to reduce inflammation in the joint of the subject and/or increase the production of anti-inflammatory factors in the joint.

Embodiment 37. The system of any one of embodiments 33-36, wherein the one or more first modules comprise the vagal nerve stimulation module, and the vagal nerve stimulation module comprises at least one instructions selected from the group consisting of sense stimulation instructions for sight, sound, touch, taste, and smell.

Embodiment 38. The system of embodiment 37, wherein the vagal nerve stimulation module comprises one or more sense stimulation instructions for sight, and the one or more sense stimulation instructions for sight include one or more instructions to view one or more figures to stimulate autonomic nervous system.

Embodiment 39. The system of embodiment 38, wherein the digital apparatus is configured to receive and display the figures.

Embodiment 40. The system of any one of embodiments 37-39, wherein the vagal nerve stimulation module comprises one or more sense stimulation instructions for sound, and the one or more sense stimulation instructions for sound include one or more instructions to hear one or more sounds to cause honor or relaxation.

Embodiment 41. The system of embodiment 40, wherein the digital apparatus is configured to receive and play the sounds.

Embodiment 42. The system of any one of embodiments 37-41, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

Embodiment 43. The system of any one of embodiments 37-42, wherein the vagal nerve stimulation module comprises one or more sense stimulation instructions for touch, and the one or more sense stimulation instructions for touch include one or more instructions for abdominal breathing.

Embodiment 44. The system of any one of embodiments 37-43, wherein the vagal nerve stimulation module comprises one or more sense stimulation instructions for taste, and the one or more sense stimulation instructions for taste include one or more instructions to eat food to stimulate digestive glands in the subject.

Embodiment 45. The system of embodiment 44, wherein the digital apparatus is configured to receive and display information related to the food.

Embodiment 46. The system of any one of embodiments 37-45, wherein the vagal nerve stimulation module comprises one or more sense stimulation instructions for smell, and the one or more sense stimulation instructions for smell include one or more instructions to stimulate digestive glands and/or to relax.

Embodiment 47. The system of embodiment 46, wherein the digital apparatus is configured to release a scent for aroma therapy.

Embodiment 48. The system of any one of embodiments 33-47, wherein the one or more first modules do not comprise one or more first instructions for a cold massage.

Embodiment 49. The system of any one of embodiments 33-48, wherein the one or more first modules comprise the joint exercise module, and the joint exercise module comprises one or more first instructions to reduce inflammation in the joint of the subject.

Embodiment 50. The system of any one of embodiments 33-49, wherein the joint of the subject is selected from the group consisting of a shoulder joint, an elbow joint, a sacroiliac joint, a hip joint, a knee joint, a joint of the wrist or hand, a joint of the ankle or foot, a neck joint, and an intervertebral joint.

Embodiment 51. The system of embodiment 49 or 50, wherein the joint exercise comprises one or more of walking, running, stretching, and bending the joint at a predetermined angle and/or speed.

Embodiment 52. The system of any one of embodiments 33-51, wherein the one or more first modules comprise the adiponectin secretion module, and the adiponectin secretion module comprises one or more first instructions to increase a secretion of adiponectin from adipose tissue.

Embodiment 53. The system of embodiment 52, wherein the one or more first instructions to increase the secretion of adiponectin from adipose tissue are selected from the group consisting of walking, biking, aerobic dance, and swimming.

Embodiment 54. The system of any one of embodiments 33-53, wherein the sensor comprises one or more selected from the group consisting of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 55. The system of embodiment 54, wherein the sensor comprises a camera, and wherein the digital apparatus is configured to detect an angle at which the subject is bending the joint.

Embodiment 56. The system of embodiment 55, wherein the adherence information comprises one or both of the angle at which the subject is bending the joint, and a difference between the angle at which the subject is bending the joint and a predetermined angle at which the subject is instructed to bend the joint.

Embodiment 57. The system of embodiment 56, wherein, if the difference between the angle at which the subject is bending the joint and the predetermined angle at which the subject is instructed to bend the joint is greater than a predetermined threshold, the system further comprises providing to the subject one or more additional instructions to adjust the angle at which the subject is bending the joint until the difference is less than or equal to the predetermined threshold.

Embodiment 58. The system of any one of embodiments 49-57, wherein the joint exercise module induces normalization of the balance between (i) sympathetic innervation on the synovial membrane, (ii) sensory innervation on the synovial membrane, and (iii) Oncostatin M production.

Embodiment 59. The system of embodiment 33-58, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

Embodiment 60. The system of embodiment 59, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

Embodiment 61. The system of embodiment 60, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.

Embodiment 62. The system of embodiment 59, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

Embodiment 63. The system of embodiment 59, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

Embodiment 64. The system of embodiment 33-63, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.

Embodiment 65. The system of embodiment 64, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

Embodiment 66. The system of embodiment 64, wherein the one or more options comprise the viewing or editing the de-identified information of the subject, and the de-identified information of the subject comprises one or more selected from the group consisting of an identification number for the subject, and the healthcare provider for the subject.

Embodiment 67. The system of embodiment 64, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

Embodiment 68. The system of embodiment 64, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

Embodiment 69. The system of embodiment 33-68, wherein the digital application further comprises a push alarm for reminding the subject complete a digital therapeutic module.

Embodiment 70. The system of embodiment 33-69, wherein the digital apparatus comprises: a digital instruction generation unit configured to generate digital therapeutic modules for preventing or ameliorating arthritis in the joint of the subject, generate digital instructions based on the digital therapeutic modules, and provide the digital instructions to the subject; and an outcome collection unit configured to collect the subject's execution outcomes of the digital instructions.

Embodiment 71. The system of embodiment 33-70, wherein the digital instruction generation unit generates the digital therapeutic modules based on biochemical factors related to the arthritis in the joint of the subject.

Embodiment 72. The system of embodiment 71, wherein the biochemical factors comprise adiponectin, Oncostatin M, IL-6, or TNF-α

Embodiment 73. The system of embodiment 33-72, wherein the digital instruction generation unit generates the digital therapeutic modules based on the inputs from the healthcare provider.

Embodiment 74. The system of embodiment 33-73, wherein the digital instruction generation unit generates the digital therapeutic modules based on information received from the subject.

Embodiment 75. The system of embodiment 74, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

Embodiment 76. The system of embodiment 33-75, wherein the digital instruction generation unit generates the digital therapeutic modules matching to imaginary parameters which correspond to the mechanism of action in and the therapeutic hypothesis for the arthritis.

Embodiment 77. The system of embodiment 76, wherein the imaginary parameters are deduced in relation to the subject's environment, behaviors, emotions, and cognition.

Embodiment 78. The system of embodiment 33-77, wherein the outcome collection unit collects the execution outcomes of the digital instructions by monitoring the subject's adherence to the digital instructions or allowing the subject to directly input the subject's adherence to the digital instructions.

Embodiment 79. The system of embodiment 33-78, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

Embodiment 80. A computing system for preventing or ameliorating arthritis in a joint of a subject in need thereof, comprising: a display configured to provide, to the subject, one or more first modules selected from the group consisting of a vagal nerve stimulation module, a joint exercise module, and an adiponectin secretion module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information.

Embodiment 81. The computing system of embodiment 80, wherein the display is further configured to provide, to the subject, one or more second modules selected from the group consisting of a vagal nerve stimulation module, a joint exercise module, and an adiponectin secretion module, each of the one or more second modules comprising the one or more second instructions for the subject to follow.

Embodiment 82. The computing system of any one of embodiments 80-81, wherein the arthritis is selected from the group consisting of osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

Embodiment 83. The computing system of any one of embodiments 80-82, wherein the subject is an adult.

Embodiment 84. The computing system of any one of embodiments 80-83, wherein the one or more first modules comprise the vagal nerve stimulation module, and the vagal nerve stimulation module comprises one or more first instructions to reduce inflammation in the joint of the subject and/or increase the production of anti-inflammatory factors in the joint.

Embodiment 85. The computing system of any one of embodiments 80-84, wherein the one or more first modules comprise the vagal nerve stimulation module, and the vagal nerve stimulation module comprises at least one instructions selected from the group consisting of sense stimulation instructions for sight, sound, touch, taste, and smell.

Embodiment 86. The computing system of embodiment 85, wherein the vagal nerve stimulation module comprises one or more sense stimulation instructions for sight, and the one or more sense stimulation instructions for sight include one or more instructions to view one or more figures to stimulate autonomic nervous system.

Embodiment 87. The computing system of embodiment 86, wherein the digital apparatus is configured to receive and display the figures.

Embodiment 88. The computing system of any one of embodiments 85-87, wherein the vagal nerve stimulation module comprises one or more sense stimulation instructions for sound, and the one or more sense stimulation instructions for sound include one or more instructions to hear one or more sounds to cause honor or relaxation.

Embodiment 89. The computing system of embodiment 88, wherein the digital apparatus is configured to receive and play the sounds.

Embodiment 90. The computing system of any one of embodiments 85-89, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

Embodiment 91. The computing system of any one of embodiments 85-90, wherein the vagal nerve stimulation module comprises one or more sense stimulation instructions for touch, and the one or more sense stimulation instructions for touch include one or more instructions for abdominal breathing.

Embodiment 92. The computing system of any one of embodiments 85-91, wherein the vagal nerve stimulation module comprises one or more sense stimulation instructions for taste, and the one or more sense stimulation instructions for taste include one or more instructions to eat food to stimulate digestive glands in the subject.

Embodiment 93. The computing system of embodiment 92, wherein the digital apparatus is configured to receive and display information related to the food.

Embodiment 94. The computing system of any one of embodiments 85-93, wherein the vagal nerve stimulation module comprises one or more sense stimulation instructions for smell, and the one or more sense stimulation instructions for smell include one or more instructions to stimulate digestive glands and/or to relax.

Embodiment 95. The computing system of embodiment 94, wherein the digital apparatus is configured to release a scent for aroma therapy.

Embodiment 96. The computing system of any one of embodiments 80-95, wherein the one or more first modules do not comprise one or more first instructions for a cold massage.

Embodiment 97. The computing system of any one of embodiments 80-96, wherein the one or more first modules comprise the joint exercise module, and the joint exercise module comprises one or more first instructions to reduce inflammation in the joint of the subject.

Embodiment 98. The computing system of any one of embodiments 80-97, wherein the joint of the subject is selected from the group consisting of a shoulder joint, an elbow joint, a sacroiliac joint, a hip joint, a knee joint, a joint of the wrist or hand, a joint of the ankle or foot, a neck joint, and an intervertebral joint.

Embodiment 99. The computing system of embodiment 97 or 98, wherein the joint exercise comprises one or more of walking, running, stretching, and bending the joint at a predetermined angle and/or speed.

Embodiment 100. The computing system of any one of embodiments 80-99, wherein the one or more first modules comprise the adiponectin secretion module, and the adiponectin secretion module comprises one or more first instructions to increase a secretion of adiponectin from adipose tissue.

Embodiment 101. The computing system of embodiment 100, wherein the one or more first instructions to increase the secretion of adiponectin from adipose tissue are selected from the group consisting of walking, biking, aerobic dance, and swimming.

Embodiment 102. The computing system of any one of embodiments 80-101, wherein the server receives the one or more second instructions from an external reviewer.

Embodiment 103. The computing system of any one of embodiments 80-102, wherein the external reviewer comprises a health professional.

Embodiment 104. The computing system of any one of embodiments 80-103, wherein the external reviewer comprises an artificial intelligence (AI).

Embodiment 105. The computing system of any one of embodiments 80-104, wherein the sensor comprises one or more selected from the group consisting of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 106. The computing system of embodiment 105, wherein the sensor comprises a camera, and wherein the digital apparatus is configured to detect an angle at which the subject is bending the joint.

Embodiment 107. The computing system of embodiment 106, wherein the adherence information comprises one or both of the angle at which the subject is bending the joint, and a difference between the angle at which the subject is bending the joint and a predetermined angle at which the subject is instructed to bend the joint.

Embodiment 108. The computing system of embodiment 107, wherein, if the difference between the angle at which the subject is bending the joint and the predetermined angle at which the subject is instructed to bend the joint is greater than a predetermined threshold, the computing system further comprises providing to the subject one or more additional instructions to adjust the angle at which the subject is bending the joint until the difference is less than or equal to the predetermined threshold.

Embodiment 109. The computing system of any one of embodiments 97-108, wherein the joint exercise module induces normalization of the balance between (i) sympathetic innervation on the synovial membrane, (ii) sensory innervation on the synovial membrane, and (iii) Oncostatin M production.

Embodiment 110. A non-transitory computer readable medium having stored thereon software instructions for preventing or ameliorating arthritis in a joint of a subject in need thereof that, when executed by a processor, cause the processor to: display, by an electronic device to the subject, one or more first modules selected from the group consisting of one or more first modules selected from the group consisting of a vagal nerve stimulation module, a joint exercise module, and an adiponectin secretion module, each of the one or more first modules comprising one or more first instructions for the subject to follow; sense, by a sensor in the electronic device, adherence by the subject to the instructions of the one or more first modules; transmit, by the electronic device, adherence information, based on the adherence, to a server; and receive, from the server, one or more second instructions based on the adherence information.

Embodiment 111. The non-transitory computer-readable medium of embodiment 110, wherein, when executed by the processor, the software instructions cause the processor to display one or more second modules selected from the group consisting of a vagal nerve stimulation module, a joint exercise module, and an adiponectin secretion module, each of the one or more second modules comprising the one or more second instructions for the subject to follow.

Embodiment 112. The non-transitory computer readable medium of embodiment 110 or 111, wherein the arthritis is selected from the group consisting of osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

Embodiment 113. The non-transitory computer readable medium of any one of embodiments 110-112, wherein the subject is an adult.

Embodiment 114. The non-transitory computer readable medium of any one of embodiments 110-113, wherein the one or more first modules comprise the vagal nerve stimulation module, and the vagal nerve stimulation module comprises one or more first instructions to reduce inflammation in the joint of the subject and/or increase the production of anti-inflammatory factors in the joint.

Embodiment 115. The non-transitory computer readable medium of any one of embodiments 110-114, wherein the one or more first modules comprise the vagal nerve stimulation module, and the vagal nerve stimulation module comprises at least one instructions selected from the group consisting of sense stimulation instructions for sight, sound, touch, taste, and smell.

Embodiment 116. The non-transitory computer readable medium of embodiment 115, wherein the vagal nerve stimulation module comprises one or more sense stimulation instructions for sight, and the one or more sense stimulation instructions for sight include one or more instructions to view one or more figures to stimulate autonomic nervous system.

Embodiment 117. The non-transitory computer readable medium of embodiment 116, wherein the electronic device is configured to receive and display the figures.

Embodiment 118. The non-transitory computer readable medium of any one of embodiments 115-117, wherein the vagal nerve stimulation module comprises one or more sense stimulation instructions for sound, and the one or more sense stimulation instructions for sound include one or more instructions to hear one or more sounds to cause honor or relaxation.

Embodiment 119. The non-transitory computer readable medium of embodiment 118, wherein the electronic device is configured to receive and play the sounds.

Embodiment 120. The non-transitory computer readable medium of any one of embodiments 115-119, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

Embodiment 121. The non-transitory computer readable medium of any one of embodiments 115-120, wherein the vagal nerve stimulation module comprises one or more sense stimulation instructions for touch, and the one or more sense stimulation instructions for touch include one or more instructions for abdominal breathing.

Embodiment 122. The non-transitory computer readable medium of any one of embodiments 115-121, wherein the vagal nerve stimulation module comprises one or more sense stimulation instructions for taste, and the one or more sense stimulation instructions for taste include one or more instructions to eat food to stimulate digestive glands in the subject.

Embodiment 123. The non-transitory computer readable medium of embodiment 122, wherein the electronic device is configured to receive and display information related to the food.

Embodiment 124. The non-transitory computer readable medium of any one of embodiments 115-123, wherein the vagal nerve stimulation module comprises one or more sense stimulation instructions for smell, and the one or more sense stimulation instructions for smell include one or more instructions to stimulate digestive glands and/or to relax.

Embodiment 125. The non-transitory computer readable medium of embodiment 124, wherein the electronic device is configured to release a scent for aroma therapy.

Embodiment 126. The non-transitory computer readable medium of any one of embodiments 110-125, wherein the one or more first modules do not comprise one or more first instructions for a cold massage.

Embodiment 127. The non-transitory computer readable medium of any one of embodiments 110-126, wherein the one or more first modules comprise the joint exercise module, and the joint exercise module comprises one or more first instructions to reduce inflammation in the joint of the subject.

Embodiment 128. The non-transitory computer readable medium of any one of embodiments 110-127, wherein the joint of the subject is selected from the group consisting of a shoulder joint, an elbow joint, a sacroiliac joint, a hip joint, a knee joint, a joint of the wrist or hand, a joint of the ankle or foot, a neck joint, and an intervertebral joint.

Embodiment 129. The non-transitory computer readable medium of embodiment 127 or 128, wherein the joint exercise comprises one or more of walking, running, stretching, and bending the joint at a predetermined angle and/or speed.

Embodiment 130. The non-transitory computer readable medium of any one of embodiments 110-129, wherein the one or more first modules comprise the adiponectin secretion module, and the adiponectin secretion module comprises one or more first instructions to increase a secretion of adiponectin from adipose tissue.

Embodiment 131. The non-transitory computer readable medium of embodiment 130, wherein the one or more first instructions to increase the secretion of adiponectin from adipose tissue are selected from the group consisting of walking, biking, aerobic dance, and swimming.

Embodiment 132. The non-transitory computer readable medium of any one of embodiments 110-131, wherein the sensor comprises one or more selected from the group consisting of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 133. The method of any one of embodiments 110-132, wherein the server receives the one or more second instructions from an external reviewer.

Embodiment 134. The method according to any one of embodiments 110-133, wherein the external reviewer comprises a health professional.

Embodiment 135. The method according to any one of embodiments 110-134, wherein the external reviewer comprises an artificial intelligence (AI).

Embodiment 136. The non-transitory computer readable medium of embodiment 135, wherein the sensor comprises a camera, and wherein the electronic device is configured to detect an angle at which the subject is bending the joint.

Embodiment 137. The non-transitory computer readable medium of embodiment 136, wherein the adherence information comprises one or both of the angle at which the subject is bending the joint, and a difference between the angle at which the subject is bending the joint and a predetermined angle at which the subject is instructed to bend the joint.

Embodiment 138. The non-transitory computer readable medium of embodiment 137, wherein, if the difference between the angle at which the subject is bending the joint and the predetermined angle at which the subject is instructed to bend the joint is greater than a predetermined threshold, the non-transitory computer readable medium further comprises providing to the subject one or more additional instructions to adjust the angle at which the subject is bending the joint until the difference is less than or equal to the predetermined threshold.

Embodiment 139. The non-transitory computer readable medium of any one of embodiments 127-138, wherein the joint exercise module induces normalization of the balance between (i) sympathetic innervation on the synovial membrane, (ii) sensory innervation on the synovial membrane, and (iii) Oncostatin M production.

Embodiment 140. The non-transitory computer readable medium of embodiment 110-139, wherein, when executed by the processor, the software instructions cause the processor to transmit a push alarm using the electronic device for reminding the subject complete a digital therapeutic module.

Embodiment 141. The non-transitory computer readable medium of embodiment 110-140, wherein the electronic device comprises: a digital instruction generation unit configured to generate digital therapeutic modules for preventing or ameliorating arthritis in the joint of the subject, generate digital instructions based on the digital therapeutic modules, and provide the digital instructions to the subject; and an outcome collection unit configured to collect the subject's execution outcomes of the digital instructions.

Embodiment 142. The non-transitory computer readable medium of embodiment 110-141, wherein the digital instruction generation unit generates the digital therapeutic modules based on biochemical factors related to the arthritis in the joint of the subject.

Embodiment 143. The non-transitory computer readable medium of embodiment 142, wherein the biochemical factors comprise adiponectin, Oncostatin M, IL-6, or TNF-α

Embodiment 144. The non-transitory computer readable medium of embodiment 110-143, wherein the digital instruction generation unit generates the digital therapeutic modules based on the inputs from a healthcare provider.

Embodiment 145. The non-transitory computer readable medium of embodiment 110-144, wherein the digital instruction generation unit generates the digital therapeutic modules based on information received from the subject.

Embodiment 146. The non-transitory computer readable medium of embodiment 145, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

Embodiment 147. The non-transitory computer readable medium of embodiment 110-146, wherein the digital instruction generation unit generates the digital therapeutic modules matching to imaginary parameters which correspond to the mechanism of action in and the therapeutic hypothesis for the arthritis.

Embodiment 148. The non-transitory computer readable medium of embodiment 147, wherein the imaginary parameters are deduced in relation to the subject's environment, behaviors, emotions, and cognition.

Embodiment 149. The non-transitory computer readable medium of embodiment 110-148, wherein the outcome collection unit collects the execution outcomes of the digital instructions by monitoring the subject's adherence to the digital instructions or allowing the subject to directly input the subject's adherence to the digital instructions.

Embodiment 150. The non-transitory computer readable medium of embodiment 110-149, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

1. A method of preventing or ameliorating arthritis in a joint of a subject, the method comprising:
providing, by an electronic device to the subject, one or more first modules selected from the group consisting of a vagal nerve stimulation module, a joint exercise module, and an adiponectin secretion module, each of the one or more first modules comprising one or more first instructions for the subject to follow;
sensing, using a sensor of the electronic device, adherence by the subject to the first instructions of the one or more first modules, wherein the electronic device transmits adherence information, based on the adherence, to a server, and receives one or more second instructions from the server based on the adherence information.
providing, by the electronic device to the subject, one or more second modules selected from the group consisting of an adiponectin secretion module, a vagal nerve stimulation module, and a joint exercise module, each of the one or more first modules comprising one or more second instructions.

2. The method of claim 1, wherein the arthritis is selected from the group consisting of osteoarthritis, rheumatoid arthritis, juvenile arthritis, spondyloarthropathy, lupus erythematosus, gout, infectious arthritis, reactive arthritis, psoriatic arthritis, fibromyalgia, and scleroderma.

3. The method of any one of claims 1-2, wherein the one or more first modules comprise the vagal nerve stimulation module, and the vagal nerve stimulation module comprises one or more first instructions to reduce inflammation in the joint of the subject and/or increase the production of anti-inflammatory factors in the joint.

4. The method of any one of claims 1-3, wherein the one or more first modules comprise the vagal nerve stimulation module, and the vagal nerve stimulation module comprises at least one instructions selected from the group consisting of sense stimulation instructions for sight, sound, touch, taste, and smell.

5. The method of any one of claims 1-4, wherein the one or more first modules comprise the joint exercise module, and the joint exercise module comprises one or more first instructions to reduce inflammation in the joint of the subject.

6. The method of any one of claims 1-5, wherein the joint of the subject is selected from the group consisting of a shoulder joint, an elbow joint, a sacroiliac joint, a hip joint, a knee joint, a joint of the wrist or hand, a joint of the ankle or foot, a neck joint, and an intervertebral joint.

7. The method of claim 5 or 6, wherein the joint exercise comprises one or more of walking, running, stretching, and bending the joint at a predetermined angle and/or speed.

8. The method of any one of claims 1-7, wherein the one or more first modules comprise the adiponectin secretion module, and the adiponectin secretion module comprises one or more first instructions to increase a secretion of adiponectin from adipose tissue.

9. The method of claim 8, wherein the one or more first instructions to increase the secretion of adiponectin from adipose tissue are selected from the group consisting of walking, biking, aerobic dance, and swimming.

10. The method of any one of claims 1-9, wherein the server receives the one or more second instructions from an external reviewer.

11. The method of any one of claims 1-10, wherein the sensor comprises one or more selected from the group consisting of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

12. The method of claim 11, wherein the sensor comprises a camera, and wherein the electronic device is configured to detect an angle at which the subject is bending the joint.

13. The method of claim 12, wherein the adherence information comprises one or both of the angle at which the subject is bending the joint, and a difference between the angle at which the subject is bending the joint and a predetermined angle at which the subject is instructed to bend the joint.

14. The method of claim 13, wherein, if the difference between the angle at which the subject is bending the joint and the predetermined angle at which the subject is instructed to bend the joint is greater than a predetermined threshold, the method further comprises providing to the subject one or more additional instructions to adjust the angle at which the subject is bending the joint until the difference is less than or equal to the predetermined threshold.

15. The method of any one of claims 5-14, wherein the joint exercise module induces normalization of the balance between (i) sympathetic innervation on the synovial membrane, (ii) sensory innervation on the synovial membrane, and (iii) Oncostatin M production.

16. The method of any one of claims 1-15, further comprising administering to the subject one or more drugs for preventing or ameliorating arthritis, wherein the one or more drugs are administered to the subject in an amount and/or frequency that is less than an amount and/or frequency prescribed to an average arthritis patient.

17. The method of any one of claims 1-16, wherein the method is performed without administering one or more drugs for preventing or ameliorating arthritis to the subject.

18. A system for preventing or ameliorating arthritis in a joint of a subject, comprising:
a digital apparatus configured to execute a digital application comprising one or more first modules for preventing or ameliorating arthritis in a joint of the subject, wherein the digital apparatus comprises a sensor for sensing adherence by the subject to a first set of instructions of the one or more first modules;
a healthcare provider portal configured to provide one or more options to a healthcare provider to perform one or more tasks to prescribe treatment for preventing or ameliorating arthritis in a joint of the subject based on information received from the digital application; and
an administrative portal configured to provide one or more options to an administrator of the system to perform one or more tasks to manage access to the system by the healthcare provider.

19. A computing system for preventing or ameliorating arthritis in a joint of a subject in need thereof, comprising:
a display configured to provide, to the subject, one or more first modules selected from the group consisting of a vagal nerve stimulation module, a joint exercise module, and an adiponectin secretion module, each of the one or more first modules comprising one or more first instructions for the subject to follow;
a sensor configured to sense adherence by the subject to the instructions of the one or more first modules;
a transmitter configured to transmit adherence information, based on the adherence, to a server; and
a receiver configured to receive, from the server, one or more second instructions based on the adherence information.

20. A non-transitory computer readable medium having stored thereon software instructions for preventing or ameliorating arthritis in a joint of a subject in need thereof that, when executed by a processor, cause the processor to:
display, by an electronic device to the subject, one or more first modules selected from the group consisting of one or more first modules selected from the group consisting of a vagal nerve stimulation module, a joint exercise module, and an adiponectin secretion module, each of the one or more first modules comprising one or more first instructions for the subject to follow;
sense, by a sensor in the electronic device, adherence by the subject to the instructions of the one or more first modules;
transmit, by the electronic device, adherence information, based on the adherence, to a server; and
receive, from the server, one or more second instructions based on the adherence information.
